SEQUENCE LISTING (10 sequences)

SEQ ID NO: 1 - protein, 22 aa, synthetic construct
LEKILVSVGATAVTPIVHHVAC

SEQ ID NO: 2 - DNA, 1332 bp, synthetic construct
  source 1..1332: mol_type = other DNA
gaagtgatactggtggagtctgggggaggcttagtgaagcctggagggtccctgaaactctcctgtgcagcctctggattcactttcagtagctatgccatgtcttgggttcgccagactccggagaagaggctggagtgggtcgcaaccattactggtggtggtacttatatttattatccagacagtgtgaaggggcgattcaccatctccagagacaatgccaagaacaccctgtacctgcaaatgagcagtctgaggtctgaggacacggccatgtatttctgtgcaaggatctacggtcgtaactactactttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatag

SEQ ID NO: 3 - protein, 443 aa, synthetic construct
EVILVESGGGLVKPGGSLKLSCAASGFTFSSYAMSWVRQTPEKRLEWVATITGGGTYIYYPDSVKGRFTISRDNAKNTLYLQMSSLRSEDTAMYFCARIYGRNYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 4 - DNA, 639 bp, synthetic construct
  source 1..639: mol_type = other DNA
caaattgttctcacccagtctccagcaatcatgtctgcatctctaggggaggagatcaccctaacctgcagtgccaactcgagtgttacttacatgcactggtaccagcagaagtcaggcacttctcccaaactcttgatttatagcacatccaacctggcttctggagtcccttctcggttcagtggcagtgggtctgggaccttttattctctcacaatcagcagtgtggaggctgaagatgctgccgattattactgccatcagtggagtagttatcggacgttcggtggaggcaccaagctggaaatccaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 5 - protein, 212 aa, synthetic construct
QIVLTQSPAIMSASLGEEITLTCSANSSVTYMHWYQQKSGTSPKLLIYSTSNLASGVPSRFSGSGSGTFYSLTISSVEAEDAADYYCHQWSSYRTFGGGTKLEIQRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 8 aa, synthetic construct
GFTFSSYA

SEQ ID NO: 7 - protein, 8 aa, synthetic construct
ITGGGTYI

SEQ ID NO: 8 - protein, 12 aa, synthetic construct
ARIYGRNYYFDY

SEQ ID NO: 9 - protein, 5 aa, synthetic construct
SSVTY

SEQ ID NO: 10 - protein, 8 aa, synthetic construct
HQWSSYRT

(19) United States
(12) Patent Application Publication
(10) Pub. No.: US 2026/0035452 A1
(43) Pub. Date: Feb. 5, 2026

(54) NOVEL INTERLEUKIN-17A (IL-17A)-DERIVED PEPTIDE AND NEUTRALIZING ANTIBODY (AB17-IPL-1)

(71) Applicant: THE UNIVERSITY OF BIRMINGHAM, Birmingham (GB)

(72) Inventors: Francesco Maione, Napoli (IT); Paolo Grieco, Castelmezzano (IT); Asif Iqbal, Solihull (GB); Maria Rosaria Bucci, Napoli (IT); Francesco Merlino, Napoli (IT); Federica Raucci, Caserta (IT); Anella Saviano, Gricigniano di Aversa (IT); Rosa Bellavita, Cardito (IT)

(73) Assignee: THE UNIVERSITY OF BIRMINGHAM, Birmingham (GB)

(21) Appl. No.: 19/100,196

(22) PCT Filed: Aug. 3, 2023
(86) PCT No.: PCT/EP2023/071542

§ 371 Date: Jan. 31, 2025

(30) Foreign Application Priority Data
Aug. 4, 2022 (IT) 102022000016722

(51) Int. Cl. C07K 16/24; A61K 38/00; A61P 17/06; C12N 5/071; C12N 15/11; C12N 15/85; G01N 33/68

(52) U.S. Cl. CPC C07K 16/244; A61K 38/00; A61P 17/06; C12N 5/0602; C12N 15/11; C12N 15/85; G01N 33/6854; G01N 33/6869; C07K 2317/24; C07K 2317/51; C07K 2317/515; C07K 2317/565; G01N 2500/20

(57) ABSTRACT

The present invention relates to the identification of novel therapeutic targets and their use in the mitigation of inflammatory pathologies. In particular, the present invention concerns the field of anti-interleukin-17A antibodies, pharmaceutical compositions comprising said antibodies and medical uses of the same. Further aspects of the invention relate to a polynucleotide encoding the anti-interleukin-17A, vectors comprising the polynucleotide and host cells incorporating said vectors. In a further aspect the invention describes a peptide for the identification of anti-interleukin-17 compounds.

### FIELD OF THE INVENTION

The present invention relates to the identification of novel therapeutic targets and their use in the mitigation of inflammatory pathologies. In particular, the present invention concerns the field of anti-interleukin-17A antibodies, pharmaceutical compositions comprising said antibodies and medical uses of the same. Further aspects of the invention relate to a polynucleotide encoding the anti-interleukin-17A, vectors comprising the polynucleotide and host cells incorporating said vectors.

In a further aspect, the invention describes a peptide for the identification of anti-interleukin-17 compounds.

### STATE OF THE ART

T helper 17 cells (Th17) were named after discovering the cytokine they produce, IL-17, whose pleiotropic activities include the induction of proinflammatory cytokines such as tumor necrosis factor alpha (TNFα), IL-1, and IL-6, as well as chemokines such as IL-8 and monocyte chemoattractant protein 1 (MCP1) in various cell types.

T cells and innate immune cells that produce IL-17 play key protective roles in immunity to fungal, bacterial, and many viral and parasitic pathogens but can also mediate damaging infection-associated immunopathology or, through the influence of genetic and environmental factors, lead to the development of autoimmune or other chronic inflammatory diseases. IL-17 produced during infection with pathogens or commensal microorganisms, although not specific for self-antigens, may indirectly precipitate or exacerbate autoimmune diseases by priming autoreactive Th17 cells. In fact, IL-17 induced by infection or during sterile inflammation may promote inflammatory responses that are central to many different pathologies, including cardiovascular and neuroinflammatory diseases, neutrophilic asthma, cytokine storms and sepsis, and IL-17 is therefore a drug target in these diseases.

Studies using IL-17 or IL-17 receptor (IL-17R) knock-out mice have highlighted a key role for this cytokine in several animal models of autoimmune diseases including collagen-induced arthritis, experimental autoimmune encephalomyelitis, experimental colitis and allergic asthma. The clinical relevance of these findings has been confirmed by a growing number of studies examining the role of IL-17 in chronic inflammatory diseases, highlighting the importance of this cytokine in autoimmunity and inflammation.

IL-17 family is composed of six members, IL-17A to IL-17F. IL-17A and F are the two members with the highest structural homology (50%) normally present as homodimers or as IL-17A/F heterodimers. Even if these cytokines share common roles, IL-17A is more powerful and active than IL-17F and its heterodimers. Other family members (except for IL-17E, also known as IL-25) are considered pro-inflammatory, although their biological roles have not been fully elucidated.

Collectively these cytokines perform their biological activities through binding with their cognate receptors. IL-17R family includes five subunits, from IL-17RA to IL-17RE. IL-17RA and RC subunits interact with IL-17A, IL-17F, and IL-17A/F, while only IL-17RA binds to IL-25, mediating both pro- and anti-inflammatory responses. Nevertheless, IL-17RA has a greater affinity with IL-17A (˜100 fold) compared to IL-17F, while an intermediate affinity for the IL-17A/F heterodimer.

The biological activity of both IL-17A and IL-17F or IL-17A/F are normally explicated due to the interaction of N- and C-terminal portions of these proteins on their own receptor/s. Liu and coll. identified specific regions of interactions between the cytokines of the IL-17 family and its receptor: regions 1 and 2, formed by the N-terminal region and central β-strands of IL-17A, respectively, that bind IL-17RA domain 1; and region 3, where the C-terminal region of IL-17A contacts the IL-17RA domain 2. Region 1 is conserved in all IL-17 cytokines; region 2 has a major binding interface between IL-17A and IL-17RA due to a specific hydrophilic interaction; region 3 is almost entirely unique to IL-17A at this site. The higher or lesser affinity of IL-17RA for IL-17A and IL-17F is attributable to multiple interactions formed between these regions and the receptor (Liu et al., 2013, “Crystal structures of interleukin 17A and its complex with IL-17 receptor” A. Nat. Commun 2013; 4:1888).

In the process of ongoing inflammation, dysregulation of IL-17A production and the binding to its receptor have been associated with several inflammatory disorders, including psoriasis, psoriatic arthritis (PsA), rheumatoid arthritis (RA), and ankylosing spondylitis (AS) and Sjögren diseases (SS). As such, this complex is an attractive target for therapeutic interventions. Indeed, some monoclonal antibodies (mAb) against IL-17A are already effective in the treatment of plaque psoriasis, PsA and AS, such as secukinumab and ixekizumab. Despite the potent blockade of cytokine signalling offered by biological therapies, many patients have only partial or transient response which is why further therapeutic modalities are required.

Drugs that antagonize inflammatory cytokines are used therapeutically to downregulate immune-mediated pathology in the mentioned conditions, although not all patients respond well to this approach. Therefore, the identification of potential novel therapeutic targets, such as the IL-17 signalling complex, may be clinically relevant for mitigating inflammatory pathology.

The need and importance is increasingly felt for the identification of a the active fragment of IL-17A in order to be able to develop anti-IL17A compounds such as neutralizing antibodies useful in the treatment of inflammatory and immune-mediated diseases.

### SUMMARY OF THE INVENTION

The problem underlying the present invention concerns making available compounds that allow to neutralize anti-IL17A. This problem is resolved by the identification of an isolated anti-interleukin-17A antibody, wherein said antibody comprises: a. a heavy chain (HC) having the amino acid sequence of SEQ ID NO: 3; and b. a light chain (LC) having the amino acid sequence of SEQ ID NO: 5.

In a second aspect, the invention relates to a polynucleotide encoding the anti-interleukin-17A antibody.

According to a third aspect, as herein described, the invention relates to a vector comprising the polynucleotide encoding the anti-interleukin-17A antibody, wherein the vector is optionally an expression vector.

In a fourth aspect, the invention concerns a host cell comprising the vector comprising the polynucleotide encoding the anti-interleukin-17A antibody, wherein said host cell is prokaryotic, eukaryotic, or mammalian.

According to a fifth aspect, the invention relates to a pharmaceutical composition comprising (i) the anti-interleukin-17A antibody or (ii) the polynucleotide as herein described, wherein the composition optionally further comprises pharmaceutically acceptable carriers and/or excipients.

According to a sixth aspect, the, present invention describes an anti-interleukin-17A antibody or a pharmaceutical composition as herein defined, for use as a medicament. In a seventh aspect, as herein described is the use of the peptide of SEQ ID NO:1, for the identification of anti-interleukin-17 compounds.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-19.

FIG. 1: shows an illustration of the Ab17-IPL-1 antibody-encoding gene (5′ and 3′ adaptors added).

FIG. 2: NIH-3T3 mouse embryonic fibroblast cells were treated with IL-17 vehicle (Ctrl), IL-17 (50 ng/ml) alone (IL-17) or peptide (FM-0410) (50 ng/ml) and supernatants were assayed by Elisa for IL-6. Data were expressed as pg/ml and presented as means±S.D. of three separate independent experiments. Statistical analysis was conducted by one-way ANOVA followed by Bonferroni's for multiple comparisons. ^{##}P≤0.01, ^{###}P≤0.005 vs Ctrl group; *P≤0.05 vs IL-17 group.

FIG. 3: Mice were treated with IL-17 vehicle (Ctrl), IL-17 (1 μg/mice) alone (IL-17) or peptide (FM-0410) (1 μg/mice) and thereafter total cell number from pouches inflammatory exudates was evaluated at 24 h. Data were expressed as millions of cells for pouch and presented as means±S.D. of n=7 mice per group. Statistical analysis was conducted by one-way ANOVA followed by Bonferroni's for multiple comparisons. ^{##}P≤0.01, ^{###}P≤0.005 vs Ctrl group; *P≤0.05 vs IL-17 group.

FIG. 4: Inflammatory supernatants obtained from the pouch cavities were assayed using a Proteome Profiler Cytokine Array for Ctrl, IL-17 and FM-0410 experimental group. Densitometric analysis is presented as heatmap. Data (expressed as INT/mm₂) are presented as means±S.D. of positive spots of three independent experiments run each with n=7 mice per group.

FIG. 5: Mice were treated with IL-17 vehicle (Ctrl), IL-17 (1 μg/mice), or peptide (FM-0410) (1 μg/mice) and inflammatory fluids were assayed by Elisa for IL-6. Data were expressed as pg/ml and presented as means±S.D. of n=7 mice per group. Statistical analysis was conducted by one-way ANOVA followed by Bonferroni's for multiple comparisons. ^{##}P≤0.01 vs CTRL group.

FIG. 6: NIH-3T3 mouse embryonic fibroblast cells were treated with IL-17 vehicle (Ctrl), IL-17 (50 ng/ml) and peptide (FM-0410, 50 ng/ml) alone or in administration with IL-17 neutralizing antibody (Ab17, 750 ng/ml) and supernatants were assayed by Elisa for IL-6. Data were expressed as pg/ml and presented as means±S.D. of three separate independent experiments. Statistical analysis was conducted by one-way ANOVA followed by Bonferroni's for multiple comparisons. ^{##}P≤0.01, ^{###}P≤0.005 vs Ctrl group; ^{£}P≤0.05 vs IL-17 group; *P≤0.05, **P≤0.01 vs respective groups.

FIG. 7. Mice were treated with FM-0410 vehicle (Ctrl), peptide (FM-0410) (1 μg/mice) alone or co-administrated with IL-17 (Ab17) (A), KC (Anti-KC) and JE (Anti-JE) (B) neutralizing antibodies (10 μg/mice) and thereafter total cell number from pouches inflammatory exudates was evaluated at 24 h. Data were expressed as millions of cells for pouch and presented as means±S.D. of n=7 mice per group. Statistical analysis was conducted by one-way ANOVA followed by Bonferroni's for multiple comparisons. ^{###}P≤0.005 vs Ctrl group; *P≤0.05, **P≤0.01 ***P≤0.005 vs FM-0410 group.

FIG. 8: NIH-3T3 mouse embryonic fibroblast cells were treated with FM-0410 vehicle (Ctrl), FM-0410 (50 ng/ml) alone or in administration with Clone #9, #12, #14 (75-7500 ng/ml), and supernatants were assayed by Elisa for IL-6. Data were expressed as pg/ml and presented as means±S.D. of three separate independent experiments. Statistical analysis was conducted by one-way ANOVA followed by Bonferroni's for multiple comparisons. ^{###}P≤0.005 vs Ctrl group; **P≤0.01; ***P≤0.005 vs FM-0410 group.

FIG. 9: Neutrophil cells were seeded into the top chamber which had a confluent HDBEC monolayer activated with TNF-α (100 U/ml) and IFN-γ (10 ng/ml) for 24 hours. The cells migration after IL-17 (10-500 ng/ml), FM-0410 (10-500 ng/ml) or N-Formylmethionine-leucyl-phenylalanine (fmlp; 10⁻⁶ M) treatment was quantified using CountBright™ Absolute Counting Beads. Data were presented as means±S.D. of n=3 healthy donors. Statistical analysis was conducted by one-way ANOVA followed by Bonferroni's for multiple comparisons. ^{#}P≤0.05, ^{##}P≤0.01, ^{###}P≤0.001, ^{####}P≤0.0001 vs Ctrl group; ⁺P≤0.05 vs IL-17.

FIG. 10: Human dermal blood endothelial cells (HDBEC) were treated with: IL-17 vehicle (Ctrl), IL-17 or FM-0410 (100 ng/ml), alone or in combination with TNF-α (100 U/ml) for 24 h. Cells were washed, gated in their totality and singlet before the identification of IL-17Rs (RA and RC) (A, B), ICAM (C) and VCAM (D) expression. Histogram values (expressed as MIF) indicate the total positive cells (A-D). In (E) are shown phase-contrast micrographs of confluent monolayers of untreated or treated (as reported before) HDBEC. Total adhesion (expressed as INT/mm², F) and % of transmigration (G) are calculated with Image Pro programme (DataCell, Finchampstead). Inflammatory supernatants from all experimental conditions were assayed by Elisa for IL-6 (H) expressed in pg/ml. Data were presented as means±S.D. of n=3 healthy donors. Statistical analysis was conducted by one-way ANOVA followed by Bonferroni's for multiple comparisons. ^{#}P≤0.05, ^{##}P≤0.01, ^{###}P≤0.001, ^{####}P≤0.0001 vs Ctrl group; *P≤0.05 vs TNF-α+IL-17 group; ^{∘}P≤0.05, ^{∘∘∘}P≤0.001, ^{∘∘∘∘}P≤0.0001 vs TNF-α group.

FIG. 11: Human fibroblasts from Resolving and RA patients were stimulated for 24 h with IL-17 (10 ng/ml) and TNF-α (100 U/ml) alone or in combination (30 minutes before) with MAB421 (10 μg/ml) or Ab17-IPL-1 (10 μg/ml) and supernatants were then assayed by Elisa for IL-6. Data were expressed as pg/ml and presented as means±S.D. of three separate independent experiments. Statistical analysis was conducted using analysis of variance with Bonferroni post-test for multiple comparisons. ^{###}P≤50.001 vs own Ctrl group; **P≤0.01 vs RA IL-17 group; ⁺P≤0.05 vs Res IL-17 group.

FIG. 12: Heavy chain, HC (A) and Light chain, LC (B) PCR amplification results: HC1-HC2: Heavy chain (molecular weight around 1 400 bp) was amplified using 2 different sets of primers to increase the chance of success. LC1-LC2: Light chain (molecular weight around 750 bp) was amplified using 2 different sets of primers to increase chance of success. MW: molecular weight standard.

FIG. 13: PCR validation after cloning. Clones 1-7 of HC (A) and clones 1-8 of LC (B) were validated and confirmed to have the correct size (around 1 400 bp and 750 bp, respectively). The clones were sequenced. MW: molecular weight standard.

FIG. 14: Ab-IPL-IL-17™ displays a protective profile in murine preclinical models of immune-mediated inflammatory diseases. To assess the neutralising activity of Ab-IPL-IL-17™, CD-1 mice were injected i.p. with 100 μg/mouse of Ab-IPL-IL-17™, MAB421 or secukinumab as positive controls. After 30 min, an i.p. injection of 10 μg/mouse of IL-17A, IL-17F or IL-17A/F heterodimer was administered. After 2 h blood was collected by intracardiac puncture and serum levels of (A) IL-17A, (B) IL-17F or (C) IL-17A/F heterodimer were quantified by Elisa. (C-F) For the evaluation of immunogenic effects CD-1 mice were injected i.p. with 100 μg of IgG1 isotype antibody (vehicle) or IL-17 neutralising antibodies (bimekizumab, secukinumab, MAB421 or Ab-IPL-IL-17™). In the selected time-point (2 h, 24 h, 72 h, 7 days, 14 days and 21 days) total IgG, IgG1, lymphocytes and platelets levels were determined by Elisa and haematological blood count test, respectively. Data are presented as mean±S.D. for n=5 mice per group. Statistical analysis was conducted by one or two-way ANOVA followed by Bonferroni's for multiple comparisons. *P≤0.05, **P≤0.01, ***P≤0.001 vs IL-17 group; ^{#}P≤0.05, ^{##}P≤0.01, ^{###}P≤0.001, ^{####}P≤0.0001 vs vehicle group.

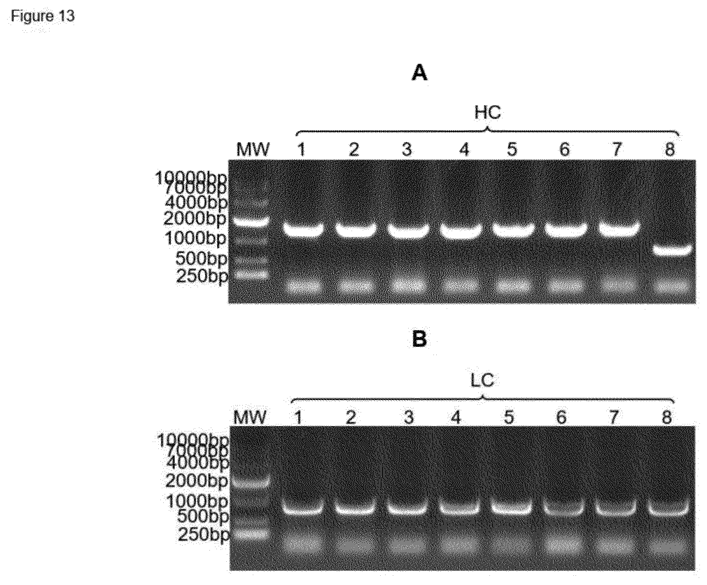

FIG. 15: Ab-IPL-IL-17™ displays a protective profile in human preclinical models of immune-mediated inflammatory diseases. Monoarthritic mice (AIA group) were therapeutically administered Ab-IPL-IL-17™ or infliximab (anti-TNF-α) on day 1 and 3. Joint inflammation was scored daily and expressed as (A) percentage of baseline joint thickness or (B) AUC. (A-B) Data are presented as mean±S.D. for n=6 mice per group. Statistical analysis was conducted by one or two-way ANOVA followed by Dunnett post-test. *P≤0.05, **p≤0.01 vs AIA group. (C) Human whole blood from IBD patients was treated with or without Ab-IPL-IL-17™ (10 μg/ml) for 4 h, after which serum IL-17 levels were assessed by Elisa assay. Data are median±interquartile ranges (min 25%, max 75%) for n=6 independent donors. Statistical analysis was conducted by one-way ANOVA followed by Bonferroni's for multiple comparisons. ^{#}P≤0.05 vs vehicle group.

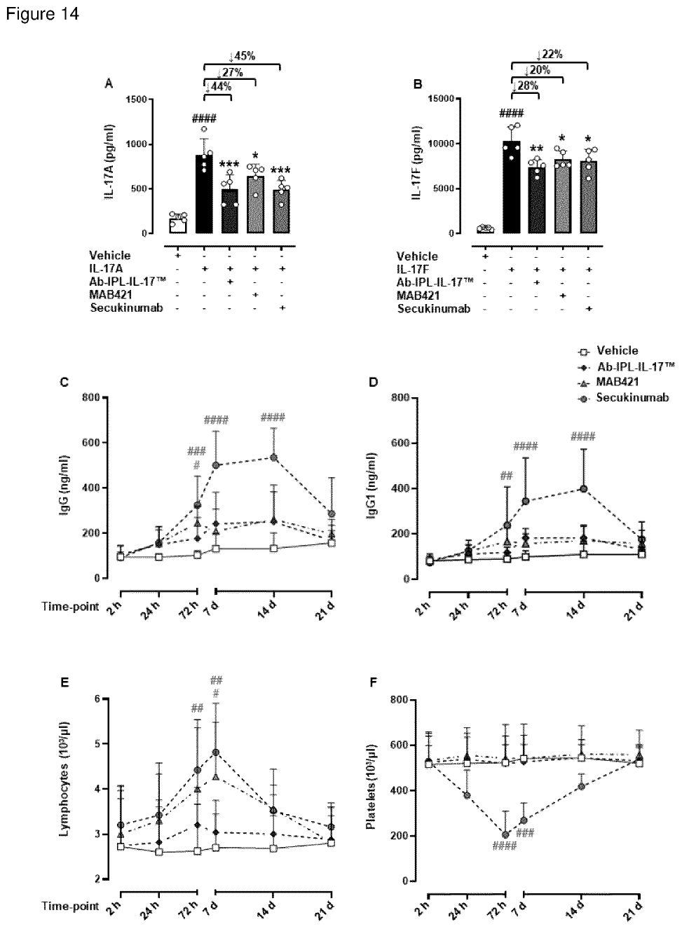

FIG. 16: Biological characterisation of a novel IL-17 neutralising antibody (Ab-IPL-IL-17). (A) To assess the biological activity of Ab-IPL-IL-17, IL-6 production was evaluated in NIH-3T3 cell supernatants following 24 hours treatment with IL-17 (50 ng/mL) or nIL-17 (50 ng/mL) alone or in combination with Ab-IPL-IL-17 (75-750 ng/mL). (B) To analyse the neutralisation effect of Ab-IPL-IL-17 on IL-17/IL-17Rs interactions, biotinylated IL17 (EC50 concentrations) and Ab-IPL-IL-17 (0-750 ng/mL) complex was co-incubated for 30 min with IL-17RA-Fc or IL-17RC-Fc prior to fluorescence being measured. (A)-(B) Data are presented as mean±SD of n=3 independent experiments. (C)-(D) Macrophages, derived from primary human CD14+ monocytes, were stimulated with LPS and IFN-γ (M1-stimuli) over 16 hours. Following differentiation, cells were treated with IL-17 vehicle, IL-17 (100 ng/mL) alone or in combination with Ab-IPL-IL-17 (10 μg/mL) for 24 hours. Supernatants from all experimental conditions were assayed by ELISA for (C) IL-6 and (D) TNF-α. (E) For the transwell chemotaxis assay, neutrophils were added to the top chamber, which had a confluent stimulated (TNF-α and IFN-γ) HDBEC monolayer. (E) Chemotactic migration to IL-17 (500 ng/mL) alone or in combination with Ab-IPL-IL-17 (10 μg/mL) was quantified using flow cytometry. (C)-(E) Data are presented as means±SD of n=3 independent healthy donors. (F) For in vivo experiment, mice were treated with IL-17 vehicle (0.5% CMC), IL-17 (1 μg/pouch) alone or in co-administration with Ab-IPL-IL-17 (10 μg/mL), and thereafter total CD45+ leucocyte numbers were quantified by flow cytometry. (F) Data are presented as means±SD of n=7 mice per group. (G) Inflammatory supernatants obtained from the pouch cavities were assayed using a Proteome Profiler Cytokine Array. Densitometric analyses are presented as a heat map indicating the most significant modulated cyto-chemokines mediators. (G) Data are presented as means±SD of positive spots of three separate independent experiments run each with n=7 mice per group pooled. (H)-(J) HDBECs were treated with IL-17 vehicle (HCl 4 mM PBS), IL-17 (100 ng/mL) plus TNF-α (100 U/mL) alone or in combination with Ab-IPL-IL-17 (10 μg/mL) for 24 hours. Phase bright PBMCs were considered (H) adherent (red arrow), whereas phase-dark were quantified as (1) transmigrated (% of adherent cells) (orange arrow). (J) Representative images of the static adhesion assay are shown (200 μm magnification). (K)-(L) VCAM-1 and ICAM-1 expression on HDBECs was quantified by flow cytometry. (H)-(L) Data are presented as means±SD of n=3 independent healthy donors. Statistical analysis was conducted by one or two-way ANOVA followed by Bonferroni's for multiple comparisons. ##p≤0.01, ###p≤0.001, ####p≤0.0001 vs vehicle group; *p≤0.05, **p≤0.01, ****p≤0.0001 vs IL-17 group; § § p≤0.01, § §§ p≤0.001, § §§ § p≤0.0001 vs nIL-17 group; +p≤0.05, ++p≤0.01, ++++p≤0.0001 vs IL-17+TNF-α group. CMC, carboxymethyl cellulose; HDBECs, human dermal blood endothelial cells; ICAM-1, intercellular adhesion molecule-1; IFN-γ, interferon gamma; IL-17, interleukin-17; LPS, lipopolysaccharide; PBMCs, peripheral blood mononuclear cells; PBS, phosphate-buffered saline; TNF-α, tumour necrosis factor α; VCAM-1, vascular cell adhesion molecule-1.

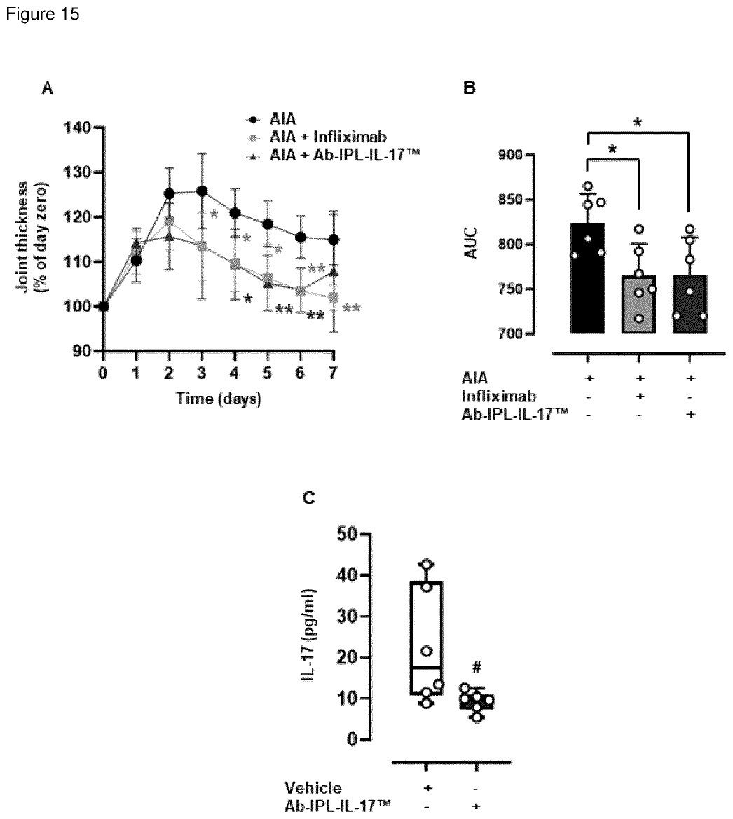

FIG. 17: (A) CD spectrum of nIL-17™ recorded in PBS buffer at 20° C., and (B) best structural model of nIL-17™ generated by PEP-FOLD 4 software.

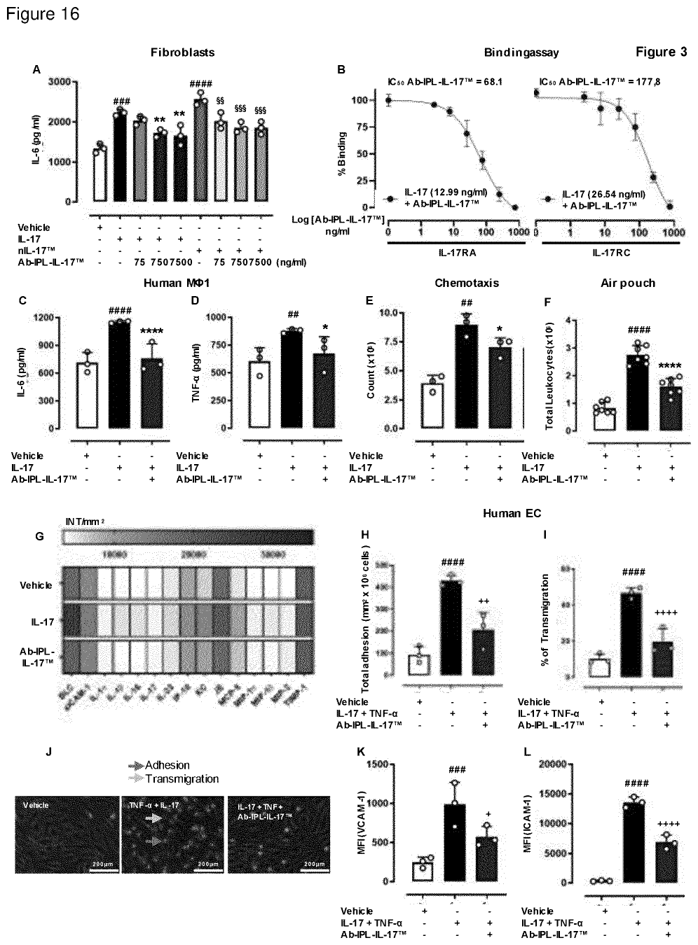

FIG. 18: (A) Docking-predicted binding mode of nIL-17™ (light blue) to IL-17RA receptor (green) (PDB id: 7ZAN). Zoom view of the binding mode of (B) the nIL-17™ C-terminal region with D2 domain, and (C) the nIL-17™ N-terminal region with D1 domain. Specifically, H18, H19, V20, and A21 of C-terminal region of nIL-17™ form H-bond interactions with S298 and Q282 of IL-17RA D2 domain. Moreover, V13 and T11 of nIL-17™ establish two H-bonds with D293 and N292 of the same receptor domain, respectively. Concerning the interactions between N-terminal region of nIL-17™ and IL-17RA D1 domain, the V8 amino acid of peptide D-turn is H-bonded to N120 of IL-17RA and, in addition, L1 of nIL-17™ N-terminal region can establish two H-bond interactions with D154 and D184 of D1 receptor domain.

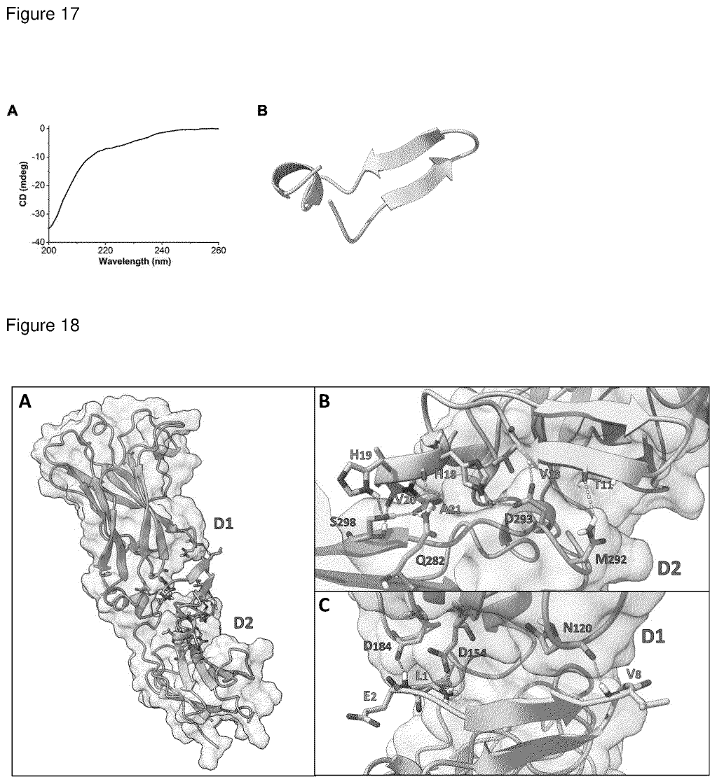

FIG. 19: (A) Docking-predicted binding mode of nIL-17™ (light blue) to IL-17RC receptor (violet) (PDB id: 7ZAN), and (B) zoom view of the binding mode highlighting the H-bond interactions. Specifically, H18, H19, and A21 of C-terminal region of nIL-17™ form H-bond interactions with W207, R284, T285, and N286 of IL-17RC D2 domain. In addition, V13 of nIL-17™ forms a H-bond to D281 side chain of the same receptor domain, as in the case of IL-17RA. The interactions established by N-terminal region of nIL-17™, L1 and E2 can establish two H-bond interactions with D193 and R195 of the linker between D1 and D2 domains, respectively.

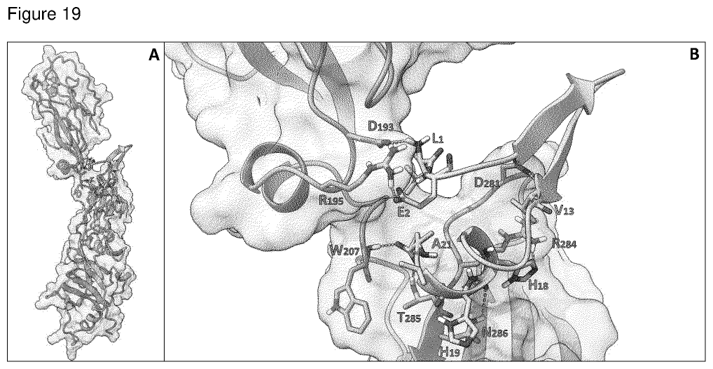

### DETAILED DESCRIPTION OF THE INVENTION

The invention herein provides an isolated antibody that binds to interleukin-17A and uses thereof. Pharmaceutical compositions, as well as a peptide for the identification of compounds that bind interleukin-17A, are also provided.

All of the currently licensed therapeutics in the IL-17-IL-17R pathway are mAbs. Some have been associated with side effects, including enhanced intestinal inflammation in patients with IBD treated with secukinumab or brodalumab (Hueber, W. et al. 2021. Secukinumab, a human anti-IL-17A monoclonal antibody, for moderate to severe Crohn's disease: unexpected results of a randomised, double-blind placebo-controlled trial. Gut 61, 1693-1700), suicidal thoughts in some patients with psoriasis treated with brodalumab (Schmidt, C. 2015. Suicidal thoughts end Amgen's blockbuster aspirations for psoriasis drug. Nat. Biotechnol. 33, 894-895), and enhanced Candida or upper respiratory tract infections in patients treated with a range of mAbs that target the IL-17-IL-17R pathway (Reich, K. et al. 2021. Bimekizumab versus Secukinumab in plaque psoriasis. N. Engl. J. Med. 385, 142-152; Warren, R. B. et al. 2021. Bimekizumab versus Adalimumab in plaque psoriasis. N. Engl. J. Med. 385, 130-141). Small molecule drugs (SMDs) have advantages not only regarding cost of production and ease of delivery but also regarding the potential of reduced infection-related side effects. Unlike biologics, which chronically block IL-17 production, SMDs are more likely to transiently blunt IL-17 production, which may break the cycle of inflammation without suppressing the protective effects of IL-17 against infection. However, off-target toxicity can be an issue with some SMDs. Therefore, there is a need for safe and effective oral bioavailable SMDs that block the IL-17-IL-17R pathway. Because of the dual role of IL-17 in protective immunity and damaging inflammation, an alternative, more targeted approach may be to exploit the host's natural immunoregulatory mechanisms that selectively suppress IL-17 responses to self-antigens or in specific diseased tissues. Selective induction of Treg cells or cell-based therapies with in vitro-expanded Treg cells have already shown proof-of-principle in animal models and, although yet to deliver major success in human clinical trials, they may provide a safe and effective approach for the treatment of autoimmune diseases in humans.

The antibody of the present invention, herein also referred to as Ab17-IPL-1, is a monoclonal antibody with many advantageous properties which result also from the fact that it was identified from the exact binding region on IL-17A and thus it has a short amino acid sequence. As will be further discussed below and demonstrated with the available experimental data, the monoclonal antibody of the invention possesses high specificity and can be used as a medicament due to its technical characteristics.

A monoclonal neutralising antibody (Ab-IPL-IL-17™) targeting nIL-17™ was developed, which effectively reversed the actions of nIL-17™ leading to reductions in chemokine, cytokine, and adhesion molecule levels on target cells, as well as reducing the inflammation infiltrate. Finally, the therapeutic efficacy of Ab-IPL-IL-17™ was compared with reference anti-IL-17 antibodies in preclinical models of IMIDs, specifically arthritis and inflammatory bowel disease (IBD). Crucially, Ab-IPL-IL-17™ exhibited significantly more neutralising activity limiting inflammation and disease progression, with lower immunogenicity and adverse haematological side effects when compared to reference antibodies. Future studies and clinical trials will need to address the varying requirements of Ab-IPL-IL17™ as an alternative biological therapy for treating patients with IMIDs.

The present invention thus concerns an isolated anti-interleukin-17A antibody, wherein said antibody comprises a. a heavy chain (HC) having the amino acid sequence of SEQ ID NO: 3; and b. a light chain (LC) having the amino acid sequence of SEQ ID NO: 5.

In a preferred aspect, the isolated anti-interleukin-17A antibody of the invention comprises 6 CDR regions, said CDR regions being:
a. a HC-CDR1 having the amino acid sequence of SEQ ID NO: 6;
b. a HC-CDR2 having the amino acid sequence of; SEQ ID NO:7;
c. a HC-CDR3 having the amino acid sequence of SEQ ID NO:8;
d. a LC-CDR1 having the amino acid sequence of SEQ ID NO:9;
e. a LC-CDR2 having the amino acid sequence of STS (Ser-Thr-Ser); and
f. a LC-CDR3 having the amino acid sequence of SEQ ID NO:10.

For the purposes of the present disclosure, each sequence has a corresponding SEQ ID NO. as follows:

SEQ ID NO. 1 corresponds to the amino acid sequence of the FM-0410 peptide:

(SEQ ID NO: 1)
Ac-LEKILVSVGATAVTPIVHHVAC.

SEQ ID NO. 2 corresponds to the DNA sequence of the Heavy chain (Identical for all clones), DNA sequence HC (1 332 bp, CDRs in bold, IgG1 constant region underlined: FR1-CDR1-FR2-CDR2-FR3-CDR3-FR4-CH-Stop):

GAAGTGATACTGGTGGAGTCTGGGGGAGGCTTAGTGAAGCCTGGAGGG
TCCCTGAAACTCTCCTGTGCAGCCTCTGGATTCACTTTCAGTAGCTAT
GCCATGTCTTGGGTTCGCCAGACTCCGGAGAAGAGGCTGGAGTGGGTC
GCAACCATTACTGGTGGTGGTACTTATATTTATTATCCAGACAGTGTG
AAGGGGCGATTCACCATCTCCAGAGACAATGCCAAGAACACCCTGTAC
CTGCAAATGAGCAGTCTGAGGTCTGAGGACACGGCCATGTATTTCTGT
GCAAGGATCTACGGTCGTAACTACTACTTTGACTACTGGGGCCAAGGC
ACCACTCTCACAGTCTCCTCAGCCAAAACGACACCCCCATCTGTCTAT
CCACTGGCCCCTGGATCTGCTGCCCAAACTAACTCCATGGTGACCCTG
GGATGCCTGGTCAAGGGCTATTTCCCTGAGCCAGTGACAGTGACCTGG
AACTCTGGATCCCTGTCCAGCGGTGTGCACACCTTCCCAGCTGTCCTG
CAGTCTGACCTCTACACTCTGAGCAGCTCAGTGACTGTCCCCTCCAGC
ACCTGGCCCAGCGAGACCGTCACCTGCAACGTTGCCCACCCGGCCAGC
AGCACCAAGGTGGACAAGAAAATTGTGCCCAGGGATTGTGGTTGTAAG
CCTTGCATATGTACAGTCCCAGAAGTATCATCTGTCTTCATCTTCCCC
CCAAAGCCCAAGGATGTGCTCACCATTACTCTGACTCCTAAGGTCACG
TGTGTTGTGGTAGACATCAGCAAGGATGATCCCGAGGTCCAGTTCAGC
TGGTTTGTAGATGATGTGGAGGTGCACACAGCTCAGACGCAACCCCGG
GAGGAGCAGTTCAACAGCACTTTCCGCTCAGTCAGTGAACTTCCCATC
ATGCACCAGGACTGGCTCAATGGCAAGGAGTTCAAATGCAGGGTCAAC
AGTGCAGCTTTCCCTGCCCCCATCGAGAAAACCATCTCCAAAACCAAA
GGCAGACCGAAGGCTCCACAGGTGTACACCATTCCACCTCCCAAGGAG
CAGATGGCCAAGGATAAAGTCAGTCTGACCTGCATGATAACAGACTTC
TTCCCTGAAGACATTACTGTGGAGTGGCAGTGGAATGGGCAGCCAGCG
GAGAACTACAAGAACACTCAGCCCATCATGGACACAGATGGCTCTTAC
TTCGTCTACAGCAAGCTCAATGTGCAGAAGAGCAACTGGGAGGCAGGA
AATACTTTCACCTGCTCTGTGTTACATGAGGGCCTGCACAACCACCAT
ACTGAGAAGAGCCTCTCCCACTCTCCTGGTAAATAG.

SEQ ID NO. 3 corresponds to the amino acid sequence of the Heavy chain, amino acid sequence HC (443aa, CDRs in bold, IgG1 constant region underlined: FR1-CDR1-FR2-CDR2-FR3-CDR3-FR4-CH-Stop):

EVILVESGGGLVKPGGSLKLSCAASGFTFSSYAMSWVRQTPEKRLEWV
ATITGGGTYIYYPDSVKGRFTISRDNAKNTLYLQMSSLRSEDTAMYFC
ARIYGRNYYFDYWGQGTTLTVSSAKTTPPSVYPLAPGSAAQTNSMVTL
GCLVKGYFPEPVTVTWNSGSLSSGVHTFPAVLQSDLYTLSSSVTVPSS
TWPSETVTCNVAHPASSTKVDKKIVPRDCGCKPCICTVPEVSSVFIFP
PKPKDVLTITLTPKVTCVVVDISKDDPEVQFSWFVDDVEVHTAQTQPR
EEQFNSTFRSVSELPIMHQDWLNGKEFKCRVNSAAFPAPIEKTISKTK
GRPKAPQVYTIPPPKEQMAKDKVSLTCMITDFFPEDITVEWQWNGQPA
ENYKNTQPIMDTDGSYFVYSKLNVQKSNWEAGNTFTCSVLHEGLHNHH
TEKSLSHSPGK-

SEQ ID NO. 4 corresponds to the DNA sequence of the Light Chain (Identical for all clones), DNA sequence LC (639 bp, CDRs in bold, Kappa constant region underlined: FR1-CDR1-FR2-CDR2-FR3-CDR3-FR4-CLKappa-Stop):

CAAATTGTTCTCACCCAGTCTCCAGCAATCATGTCTGCATCTCTAGGG
GAGGAGATCACCCTAACCTGCAGTGCCAACTCGAGTGTTACTTACATG
CACTGGTACCAGCAGAAGTCAGGCACTTCTCCCAAACTCTTGATTTAT
AGCACATCCAACCTGGCTTCTGGAGTCCCTTCTCGGTTCAGTGGCAGT
GGGTCTGGGACCTTTTATTCTCTCACAATCAGCAGTGTGGAGGCTGAA
GATGCTGCCGATTATTACTGCCATCAGTGGAGTAGTTATCGGACGTTC
GGTGGAGGCACCAAGCTGGAAATCCAACGGGCTGATGCTGCACCAACT
GTATCCATCTTCCCACCATCCAGTGAGCAGTTAACATCTGGAGGTGCC
TCAGTCGTGTGCTTCTTGAACAACTTCTACCCCAAAGACATCAATGTC
AAGTGGAAGATTGATGGCAGTGAACGACAAAATGGCGTCCTGAACAGT
TGGACTGATCAGGACAGCAAAGACAGCACCTACAGCATGAGCAGCACC
CTCACGTTGACCAAGGACGAGTATGAACGACATAACAGCTATACCTGT
GAGGCCACTCACAAGACATCAACTTCACCCATTGTCAAGAGCTTCAAC
AGGAATGAGTGTTAG

SEQ ID NO. 5 corresponds to the amino acid sequence of the Amino acid sequence LC (212aa, CDRs in bold, Kappa constant region underlined: FR1-CDR1-FR2-CDR2-FR3-CDR3-FR4-CLKappa-Stop):

QIVLTQSPAIMSASLGEEITLTCSANSSVTYMHWYQQKSGTSPKLLIY
STSNLASGVPSRFSGSGSGTFYSLTISSVEAEDAADYYCHQWSSYRTF
GGGTKLEIQRADAAPTVSIFPPSSEQLTSGGASVVCFLNNFYPKDINV
KWKIDGSERQNGVLNSWTDQDSKDSTYSMSSTLTLTKDEYERHNSYTC
EATHKTSTSPIVKSFNRNEC

SEQ ID NO: 6 corresponds to the amino acid sequence of the HC-CDR1 region.

SEQ ID NO: 7 corresponds to the amino acid sequence of the HC-CDR2 region.

SEQ ID NO: 8 corresponds to the amino acid sequence of the HC-CDR3 region.

SEQ ID NO: 9 corresponds to the amino acid sequence of the LC-CDR1 region.

The amino acid sequence of the LC-CDR2 having the amino acid sequence of STS (Ser-Thr-Ser).

SEQ ID NO: 10 corresponds to the amino acid sequence of the LC-CDR3 region.

Preferably the isolated anti-interleukin-17A antibody of the invention is a monoclonal antibody, a chimeric antibody and/or is humanized or human.

The main objective of humanization process is to reduce antibodies immunogenicity in order to improve tolerance in humans and improve their biophysical properties.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies.

Antibodies may be prepared by different techniques. For example, monoclonal antibodies may be purified from cells that naturally express them, such as hybridoma cells, or produced in recombinant expression system both from the mammalian system or prokaryotes (e.g. Escherichia Coli). More recently, fragment antibodies have been introduced in clinical practice. Indeed, fragment antibodies are emerging as great tools in imaging and diagnostics because they are capable of detecting cellular proteins with high affinity and specificity. Antibody fragments include, but not limited to: Fab, F(ab′)2, single chain antibodies, nanobodies, diabodies, triabodies, tetrabodies, and domain antibodies. They can be easily linked to radioisotopes, fluorescent molecules or enzymes that tag specific biomarkers in patients. They also have a shorter half-life in the body which results in faster clearance and may result in fewer risks of side effects from potentially invasive diagnostic agents. Where desired the affinity of the monoclonal antibody or fragment antibody according to the invention, containing one or more of CDRs above-mentioned, can be improved by affinity maturation procedures. More preferably the isolated anti-interleukin-17A antibody of the invention binds to the peptide of SEQ ID NO:1.

The advantageous properties of the antibody of the present invention will be apparent in the experimental section.

In particular, the anti-IL17A antibody herein described (Ab17-IPL-1) is a monoclonal antibody and thus exhibits a high specificity for neutralizing anti-IL17A compared to polyclonal antibodies.

Furthermore, Ab17-IPL-1 has the advantage of having a short sequence compared to other known anti-IL17A monoclonal antibodies (more or less 10 times in terms of AA related antigen sequence) and this characteristic allows to avoid non-specific binding which can result from the longer amino acid sequence. In addition to the above, a shorter sequence could, most likely, result in lower production costs and manufacturing advantages. This aspect is detrimental for future clinical application.

In a second aspect, the invention relates to a polynucleotide encoding the anti-interleukin-17A antibody.

According to a third aspect, as herein described, the invention relates to a vector comprising the polynucleotide encoding the anti-interleukin-17A antibody, wherein the vector is optionally an expression vector.

In a fourth aspect, the invention concerns a host cell comprising the vector comprising the polynucleotide encoding the anti-interleukin-17A antibody, wherein said host cell is prokaryotic, eukaryotic, or mammalian.

According to a fifth aspect, the invention relates to a pharmaceutical composition comprising (i) the anti-interleukin-17A antibody or (ii) the polynucleotide as herein described, wherein the composition optionally further comprises pharmaceutically acceptable carriers and/or excipients.

A pharmaceutical composition may optionally contain other active ingredients. The term “carrier” refers to a vehicle, excipient, diluents, or adjuvant with which the therapeutic or active ingredient is administered. Any carrier and/or excipient suitable for the form of preparation desired for administration is contemplated for use with the strains/wall/postbiotic disclosed herein.

The carrier may take a wide variety of forms depending on the form of preparation desired for administration, e.g. oral or parenteral, including intravenous. In preparing the compositions for oral dosage form, any of the usual pharmaceutical media may be employed, such as, for example, water, glycols, oils, alcohols, flavouring agents, preservatives, colouring agents and the like in the case of oral liquid preparations, such as, for example, suspensions, elixirs and solutions; or carriers such as starches, sugars, microcrystalline cellulose, diluents, granulating agents, lubricants, binders, disintegrating agents, and the like in the case of oral solid preparations such as, for example, powders, hard and soft capsules and tablets, with the solid oral preparations being preferred over the liquid preparations.

According to a sixth aspect the present invention describes an anti-interleukin-17A antibody or a pharmaceutical composition as herein defined, for use as a medicament.

In particular, the anti-interleukin antibody of the invention may be used in the treatment of inflammation, autoimmune diseases and for treating the dysregulation of IL-17A production, which has been associated with several inflammatory and autoimmune disorders, including psoriasis, psoriatic arthritis (PsA), rheumatoid arthritis (RA), and ankylosing spondylitis (AS).

In a seventh aspect, as herein described is a peptide having the amino acid sequence of SEQ ID NO: 1 and the use of the peptide of SEQ ID NO:1 for the identification of anti-interleukin-17 compounds.

The peptide of SEQ ID NO:2 is a short peptide fragment corresponding to the active region of full-length IL-17. As will be evident from the Examples, we have defined the in vitro biological activity of the peptide of SEQ ID NO:1 in terms of IL-6 production from 3T3 cell line. The peptide was identified in an in vivo model of inflammation and allowed to identify a new neutralizing antibody against the IL-17 protein useful in the treatment of inflammatory and autoimmune-based diseases.

Interleukin (IL) 17s cytokines (IL-17A, II-17F and heterodimer IL-17A/F) are key drivers of inflammation that are functionally dysregulated in several human IMIDs, such as rheumatoid arthritis (RA), psoriasis and inflammatory bowel disease (IBD).

In this study, the ‘essential’ amino acid sequence (nIL-17) responsible for IL-17A/F biological activity in both mouse and human were identified, and a novel antibody (Ab-IPL-IL-17) that specifically targets the active nIL-17 peptide sequence and has utility for understanding IL-17A/F biology/pathogenesis in mouse/human was generated.

Ab-IPL-IL-17 is as effective as reference anti-IL-17 antibodies in reducing inflammatory processes, in preclinical models of IMIDs and in human clinical samples from IBD and RA. Importantly, Ab-IPL-IL-17 exhibited, in mice, significantly more neutralising activity limiting inflammation and disease progression, with lower immunogenicity and adverse haematological side effects when compared with reference antibodies. Various embodiments and aspects of the present invention as delineated hereinabove and as claimed in the claims section below find experimental support in the following Examples section.

### EXAMPLES

### Example 1: Peptide Synthesis

We synthesized the peptide of the present invention, said peptide was called FM-0410 and has the following amino acid sequence: Ac-LEKILVSVGATAVTPIVHHVAC (SEQ ID NO:1)

Methods: The synthesis of the peptide was performed by using the ultrasound-assisted solid-phase peptide synthesis (US-SPPS), via the Fmoc/tBu orthogonal protection strategy. The peptide was assembled on a 2-chlorotrityl chloride (2-CTC) (0.1 mmol from 1.70 mmol/g as loading substitution) resin. The resin was first swollen in DMF on an automated shaker for 30 min at rt. Then, a solution of Fmoc-Ala (0.1 mmol) and DIPEA (0.2 mmol) in anhydrous DMF was added to the resin and the resulting suspension was shaken overnight at room temperature; the residual chloride reactive groups were then capped by adding a previously mixed solution of DIEA/DCM/MeOH (10:85:5) and by gently shaking for 1 h. Subsequently Fmoc-deprotection (by 20% piperidine in DMF, 0.5+1 min treatments) and coupling (by HBTU/HOBt as activating/additive agents, 5 min treatment) were cyclically performed by ultrasonic irradiation until to accomplish the resin-bound target peptide sequence. Each step was monitored by Kaiser or chloranil tests as colorimetric assays, used for the detection of solid-phase bound primary and secondary amines, respectively. The final cleavage was carried out using a cocktail of TFA/TIS/H₂O, for 3 h at rt, and crudes were purified by reverse-phase preparative HPLC. Peptide examined for biological activity was purified to >99%, and the exact molecular weight was confirmed by LC-MS analysis.

### Example 2: In Vitro Model

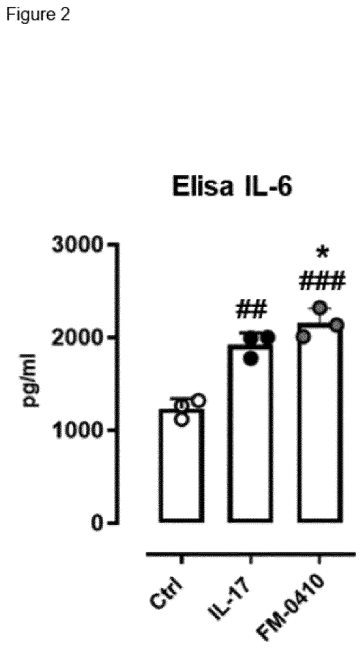

In order to assess the biological activity of synthesized peptide, we carried out in vitro studies using NIH-3T3 mouse embryonic fibroblast cells. Stimulation of fibroblasts with IL-17 induced a significant increase in IL-6 release compared to control group. Interestingly the administration of FM-0410 showed a significant, and more abundant, release of IL-6 compared to IL-17-treated group (FIG. 2).

With this in vitro system we measured IL-17 activity by its ability to induce IL-6 secretion by NIH-3T3 mouse embryonic fibroblast cells.

### Example 3: In Vivo Air Pouch Model

Only few studies have shown that IL-17 induces polymorphonuclear (PMN) infiltration in cavities such as the peritoneum or the lung. In our experiments, the inflammatory response induced by IL-17 in the air pouch was much greater in terms of both cellular infiltrate and cytokine production. Without being bound to any theory, this might be explained by the specific relevance of IL-17 in a pre-inflamed tissue and a ‘primed’ cellular microenvironment to exert its full potential. It should be in fact considered that both the peritoneum and lung are sites under immunosurveillance and, as such, under continuous control of the adaptive and innate immune systems. Conversely, the air pouch is a newly formed structure that originates as result of an unrecognized (and by day 6 inflamed) tissue reorganization. Seminal studies which utilized the air pouch have suggested that the inflammatory response in this model partially resembles the inflammatory settings found in the synovium of RA patients. Synovial fluids contain large numbers of PMNs and T cells; it is believed their presence drives destruction and erosion of the joint. The IL-17 produced locally probably by activated memory T cells as well as by neutrophils might be the key factor for exacerbation of local inflammatory circuits.

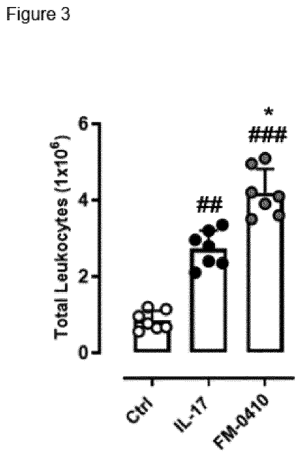

We knew from our previous studies (Maione et al., 2009 “Interleukin 17 sustains rather than induces inflammation”. Biochem Pharmacol 2009; 77, 878-87; Maione et al., 2018 “Repetitive Exposure of IL-17 into the murine air pouch favors the recruitment of inflammatory monocytes and the release of IL-16 and TREM-1 in the inflammatory fluids”, Front Immunol 2018; 2752) that a single administration of IL-17 (1 μg/pouch) into a 6-day old air pouch causes a transient infiltration of leukocytes by 4 h, which peaks at 24 h and then declines by 48 h. For these reasons we selected 24 h as our specific experimental time point. To test the potential responsive action of peptide at this local site of inflammation, we administered peptide (FM-0410) at dose of 1 μg/mice and compared its effect next to prototype peptide IL-17. Consistent with our previous findings, 24 h after IL-17 administration mice showed significant differences in the number of inflammatory leukocytes compared to control group. Interestingly, mice injected with peptide FM-0410 showed a marked increase in inflammatory infiltrates compared to control but also to IL-17-treated mice. (FIG. 3).

Specifically, dorsal air pouches were prepared by injection of 2.5 ml of air on day 0 and day 3 as previously described (Maione et al., 2009). On day 6, mice received the following treatments: i) CTRL 0.25 ml of 0.5% carboxymethyl cellulose (CMC); ii) IL-17 (1 μg) in 0.25 ml of 0.5% CMC; iii) FM-0410 (1 μg) in 0.25 ml of 0.5% CMC; iv) FM-0410+IL-17 neutralizing antibody (Ab17; 10 μg) in 0.25 ml of 0.5% CMC; v) FM-0410+KC neutralizing antibody (Anti-KC; 10 μg) in 0.25 ml of 0.5% CMC; vi) FM-0410+JE neutralizing antibody (Anti-JE; 10 μg) in 0.25 ml of 0.5% CMC. Mice were sacrificed after 24 h from the injection and air pouches washed thoroughly with 2 ml of PBS containing 50 U/ml heparin and 3 mM EDTA. Lavage fluids were centrifuged at 220×g for 10 min at 4° C. to separate the exudates from the recruited cells. Inflammatory exudates were collected and measured to evaluate the level of inflammatory cyto-chemokines. Cell number was determined by TC20 automated cell counter (Bio-Rad, Milan, Italy) using Bio-Rad's TC20 automated cell counter uses disposable slides, TC20 trypan blue dye (0.4% trypan blue dye w/v in 0.81% sodium chloride and 0.06% potassium phosphate dibasic solution) and a CCD camera to count cells based on the analyses of capture images (Maione et al., 2018).

Materials: recombinant mouse IL-17, IL-17 (Ab17), KC (Anti-KC) and JE (Anti-JE) neutralizing antibodies were purchased from R&D System (Milan. Italy).

### Example 4: Elisa and Elisa Spot Assays

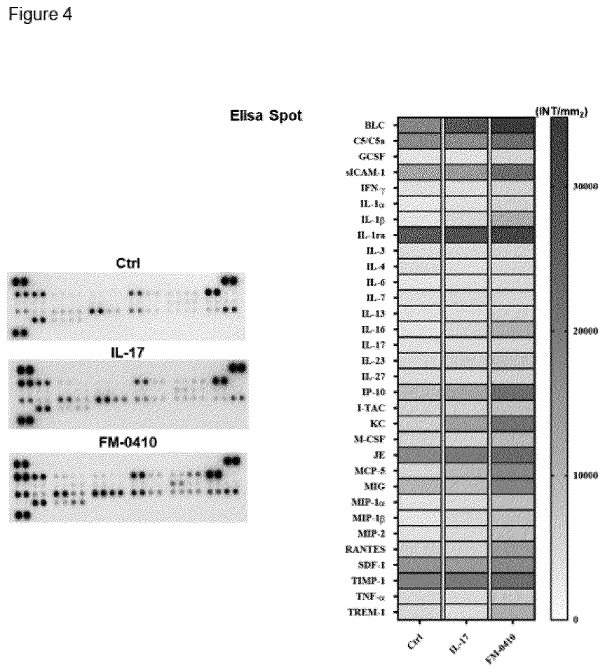

An unbiased approach (pre-made protein array) based on profiling cytokines and chemokines present in the inflammatory fluids was used. As shown in FIG. 4 the pouch fluid obtained from IL-17-injected mice showed a significant increase of cyto-chemokines profile compared to ctrl group. When comparing pouch fluids from FM-0410-treated group to IL-17 group, we observed a selective upregulation in a range of mediators. Specifically, densitometric analysis revealed that FM-0410 treated group had a specific modulation, in the following factors: B lymphocyte chemoattractant (BLC), complement component 5a (C5a), soluble intercellular adhesion molecular-1 (sICAM-1), IL-1β, IL-16, interferon γ-induced protein-10 (IP-10), keratinocyte chemoattractant (KC), macrophage colony-stimulating factor (MCSF), junctional epithelium (JE), monocytes chemoattractant protein-5 (MCP-5), monokine induced by interferon γ (MIG), macrophage inflammatory proteins (MIPs), regulated on activation, normal T cell expressed and secreted (RANTES), metallopeptidase inhibitor-1 (TIMP-1) and triggering receptor expressed on myeloid cells-1 (TREM-1) compared to IL-17 group.

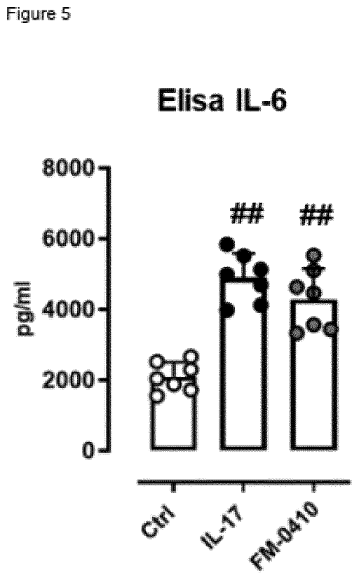

Moreover, the levels of IL-6 were quantified in pouch fluids. Administration of IL-17 and FM-0410 was correlated with an increased level of IL-6 compared to control group (FIG. 5), confirming in this mouse model of inflammation the biological effects observed in the preliminary in vitro experiments.

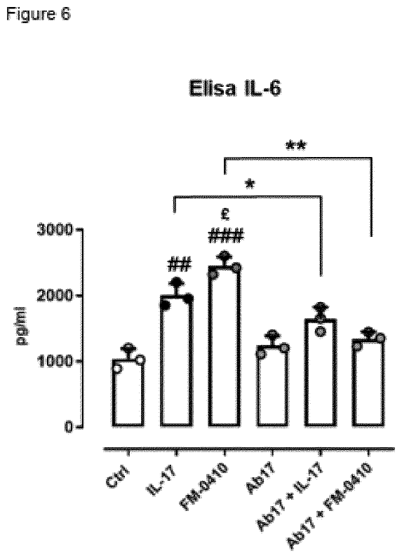

Further in vitro studies were performed, using NIH-3T3 mouse embryonic fibroblast cells, to test the potential neutralizing effect of Ab17 on Peptide FM-0410. Stimulation of fibroblast with IL-17 and FM-0410 induced a significant increase (as shown in both FIGS. 2 and 6) in IL-6 release compared to control group, reversed after administration of IL-17 neutralizing antibody (Ab17) (FIG. 6).

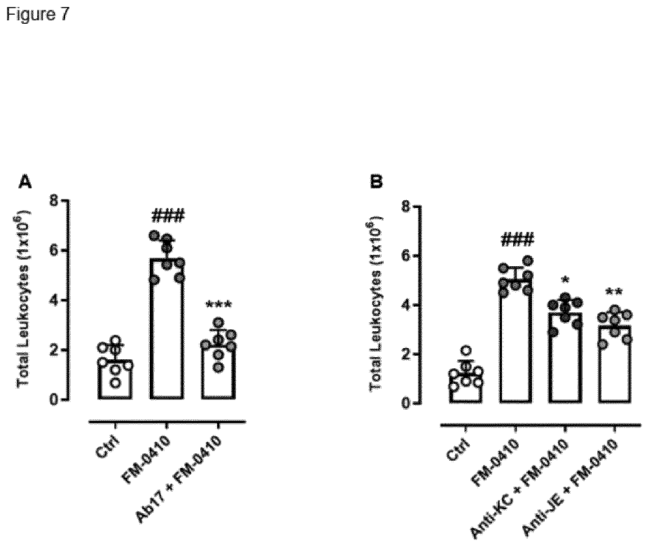

These experiments were confirmed and amplified by an additional in vivo experiment. Peptide FM-0410 was administered at dose of 1 μg/mice at this local site of inflammation and compared its effect post administration with IL-17 (Ab17), KC (Anti-KC) and JE (Anti-JE) neutralizing antibodies. Consistent with our previous findings, mice injected with peptide FM-0410 showed a marked increase in inflammatory infiltrates compared to control and IL-17-treated mice, which was reduced after neutralizing antibodies administration (FIG. 7).

Methods: The levels of IL-6 in the cellular and air pouch inflammatory supernatants at 24 h were measured using commercially available enzyme-linked immunosorbent assay kit (ELISA kit, R&D System, Milan, Italy) according to the manufacturer instructions. Briefly, 100 μl of supernatants, diluted standards, quality controls, and dilution buffer (blank) were applied on a plate with the monoclonal antibody for 2 h. After washing, 100 μl of biotin-labelled antibody was added, and incubation continued for 1 h. The plate was washed and 100 μl of the streptavidin-HRP conjugate was added and the plate was incubated for a further 30 min period in the dark. The addition of 100 μl of the substrate and stop solution represented the last steps before the reading of absorbance (measured at 450 nm) on a microplate reader. Antigen levels in the samples were determined using a standard curve and expressed as pg/ml. For cyto-chemokines protein array equal volumes (1.5 ml) of pouch inflammatory fluids in all described experimental conditions were incubated with the precoated proteome profiler array membranes according to the manufacturer's instructions. Dot plots were detected by using the enhanced chemiluminescence detection kit and Image Quant 400 GE Healthcare software (GE Healthcare, Italy) and successively quantified using GS 800 imaging densitometer software (Biorad, Italy) as previously described (Maione et al., 2018).

Materials: IL-6 Elisa kit, proteome profiler mouse cytokine array kit, were purchased from R&D System (Milan. Italy).

### Example 5: Monoclonal Antibodies Development

The aim of the procedure was the synthesis of a novel and more specific neutralizing antibody targeting a specific (and more active) portion of IL-17A which guarantees a more prominent and focused therapeutic neutralizing activity against this pro-inflammatory cytokine. For this purpose, we performed an in vitro study, using NIH3T3 mouse embryonic fibroblast cells, with the best clones obtained with the immunization protocol (at the 5th immunization process; Clone #9, #12, #14) that were used for the final production of the antibody.

The first selected concentration was based on the typical concentration used for IL-17 neutralizing antibody (Ab17), normally tested at 750 ng/ml. Considering that at this stage it was not possible to know the concentration of the supernatants because in culture supernatants other proteins (like serum proteins) were present, a protein determination was performed on clone supernatants, to assess the potential concentration of clones. Since it is only a potential concentration, we decided to test the clones at the reference concentration used for Ab17 (750 ng/ml) and at the 10-fold smaller (75 ng/ml) and 10-fold larger (7500 ng/ml) concentrations.

Our results showed that, in an almost comparable manner, all three clones had capability to revert IL-6 release at the highest dose (7500 ng/ml), while only clone #12 manages to significantly reduce cytokine release already at a lower concentration (750 ng/ml, comparable to that of Ab17).

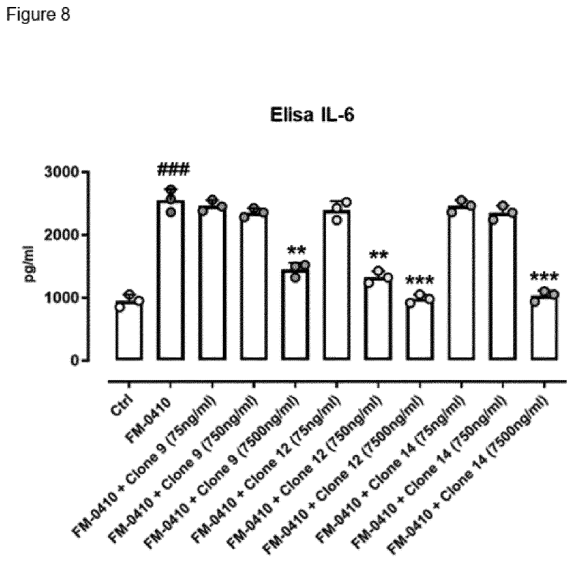

At 75 ng/ml clones weren't able to reverse the effect induced by the administration of FM-0410 peptide (FIG. 8).

Based on obtained results, the antibody production was continued starting from Clone #12.

Methods: The process of monoclonal antibody production involves multiple steps:
Step I: peptide synthesis FM-0410 (Ac-LEKILVSVGATAVTPIVHHVAC) (SEQ ID NO:1);
Step II: immunizations of 5 mice with peptide using an optimized protocol of 51-79 days (4-6 injections until optimal immune response) and immune response control such as bleedings and titers tests (ELISA against the peptide);
Step III: fusion of spleen cells from best mouse with a mouse myeloma cell line, hybridomas culture in selective medium, screenings of selection at polyclonal stage of specific hybridomas against the peptide in ELISA (around 10×96-well plates) and shipment of best clones supernatant samples for our tests;
Step IV: sub-cloning of best 2 parental clones, 2-3 rounds of limiting dilutions and screening against the peptide in ELISA, isotype determination and cell cryopreservation with preparation of vials from the best final clone;
Step V: production of the best final clone, purification by ProteinA/G (PBS pH 7.5, azide-free, sterilized by membrane filtration) and QC by SDS-PAGE plus ELISA. Table 1 summarizes the immunization protocol. The monoclonal antibody development was carried out by ProteoGenix SAS, and clone results relate to supernatants sent by the company post step III and after 5th immunization.

TABLE 1
Summary of the Immunization protocol

| Day | |
|---|---|
| 1 | 1^{st} Immunization |
| 14 | 2^{nd} Immunization |
| 28 | 3^{rd} Immunization |
| 35 | ELISA test |
| 42 | 4^{th} Immunization |
| 49 | ELISA test |
| 51 | Fusion if final boost at D42 |
| 56 | 5^{th} Immunization |
| 63 | ELISA test |
| 65 | Fusion if final boost at D56 |
| 70 | 6^{th} Immunization |
| 77 | ELISA test |
| 79 | Fusion if final boost at D70 |

### Example 6: FM-0410 has a Prominent Chemotactic Activity

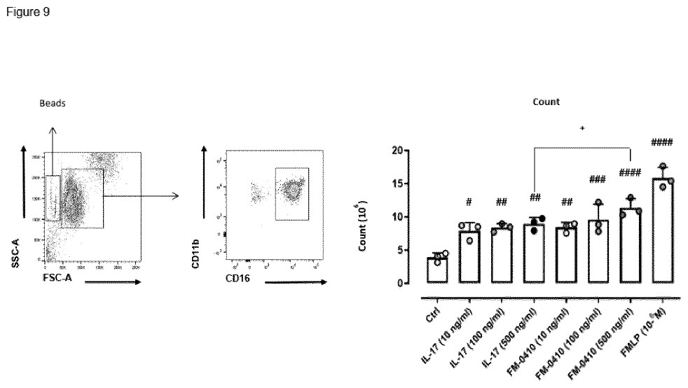

IL-17 (10-500 ng/ml) and, even more, FM-0410 displayed a concentration-dependent chemotactic effect compared to the Control group (Ctrl) as shown in FIG. 9. Notably, these activities were almost similar to positive control fmlp (10⁻⁶ M) when both proteins were tested at concentration of 500 ng/ml.

### Methods:

Human dermal blood endothelial cells (HDBEC): Primary human dermal blood endothelial cells (HDBECs) were purchased from PromoCell and cultured in the manufacturer's recommended endothelial cell growth medium MV (PromoCell, Heidelberg, Germany). HDBECs were seeded onto 12-well tissue culture plates after 4 passages at a seeding density yielding confluent monolayers. HDBEC monolayers were washed in endothelial cell growth medium MV warmed to 37° C. and stimulated with IL-17 or FM-0410 (100 ng/ml) alone or in a combination with TNF-α (100 U/ml) for 24 h at 37° C. Cells were stimulated for 24 h, before staining with IL-17Rs (RA or RC) or ICAM/VCAM adhesion receptors using a Flow cytometry assay. Supernatants were collected and measured to evaluate IL-6 or TNF-α levels.

Neutrophil isolation: Blood was taken according to local research ethics committee approval (QMERC2014/61). Informed consent was provided according to the Declaration of Helsinki. All blood samples were collected on the day of experiments from healthy donors and immediately used. Each condition has 3 biological replicates (3 different donors). Neutrophils were isolated using a double density histopaque gradient and hypotonic lysis as described previously.

Transwell chemotaxis assay: Chemotaxis was assessed using a transwell assay. IL-17 (10-500 ng/ml), FM-0410 (10-500 ng/ml) or N-Formylmethionine-leucyl-phenylalanine as a positive control (fmlp; 10⁻⁶ M) (final volume 700 μl) in M199 media were added to the bottom well of Transwell-24 permeable support with 3.0 μm pores (Corning, NY, USA). 2×10⁵ neutrophils in a final volume of 200 μl were added to the top chamber which had a confluent HDBEC monolayer activated with TNF-α (100 U/ml) and IFN-γ (10 ng/ml) for 24 hours. After 2 hours at 37° C., neutrophils were collected from the bottom wells and quantified by flow cytometry using CountBright™ Absolute Counting Beads (Thermofisher, Rugby, UK) as previously described.

### Example 7: FM-0410 Treatment Promotes Endothelial Adhesion and Transmigration

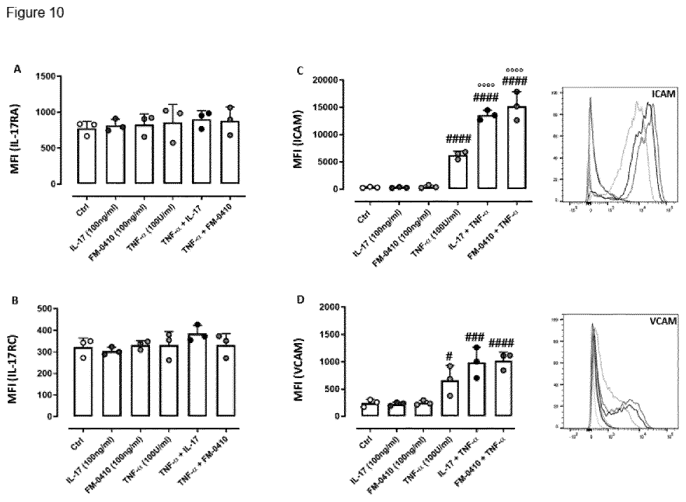

Co-stimulation of endothelial cells with IL-17 (100 ng/m) or IL-17 full length derived-peptide (FM-0410, 100 ng/ml) in combination with TNF-α (100 U/ml) induces the upregulation of both VCAM and ICAM expression (FIG. 10 C-D). No effects were noticed for both IL-17RA and RC receptors (FIG. 10 A-B). To test the effect of the FM-0410 peptide (IL-17-derived) on a static adhesion assay, hPBMC were co-cultured with cytokine-stimulated HDBEC monolayers. Our results showed that co-treatment with IL-17 or, even more, FM-0410 (plus TNF-α) caused a significant increase (FIG. 2E) in terms of total number of adhered and transmigrated hPBMC (FIG. 10 F-G). Subsequently, IL-6 levels were monitored as marker of cell activation, and our results revealed an upregulation of this cytokine after peptide co-stimulation (FIG. 10 H).

### Methods:

Flow Cytometry: HDBEC, collected after 24 h of treatment, were washed with PBS without Ca²⁺ and Mg²⁺ containing 25 mM lactose for 20 min at room temperature with occasional mixing. Cells were, then, incubated with FcR blocking agents (Miltenyi) in PBS without Ca²⁺ and Mg²⁺ containing lactose before staining cells with antibodies IL-17 Receptor A (1:100; clone 133617) and IL-17 Receptor C (1:100; clone 309822). Moreover, HDBEC cells were also stained with ICAM (BBIG-V3; 1:50, APC, BD Biosciences) and VCAM (3E2; 1:50, FITC, BD Biosciences). Protein expression was analysed by flow cytometry on a Dako CyAn (Beckman Coulter, High Wycombe, U.K.), and data were analysed using MRFIow and FlowJo software operation. The unspecific binding of antibodies was quantified by using corresponding isotype controls.

Elisa assay: The levels of IL-6 at 24 h were measured using commercially available enzyme-linked immunosorbent assay kits (ELISA kit, R&D System, Milan, Italy) according to the manufacturer's instructions. Briefly, 100 μl of supernatants, diluted standards, quality controls, and dilution buffer (blank) were applied on a plate with the monoclonal antibody for 2 h. After washing, 100 μl of biotin-labeled antibody was added and incubation continued for 1 h. The plate was washed and 100 μl of the streptavidin-HRP conjugate was added, and the plate was incubated for a further 30 min period in the dark. The addition of 100 μl of the substrate and stop solution represented the last steps before the reading of absorbance (measured at 450 nm) on a microplate reader. Antigen levels in the samples were determined using a standard curve and expressed as pg/ml.

Human peripheral blood mononuclear cells (hPBMC): Blood was collected from healthy donors with written and verbal informed consent and approval from the University of Birmingham Local Ethical Review Committee (ERN_18-0382). Human peripheral blood mononuclear cells (hPBMCs) were isolated as previously described (Riedhammer et al 2016).

Human PBMC transendothelial migration assay: Prior to beginning the assay, HDBEC monolayers were washed with 37° C. medium 199 (Thermo Fisher Scientific) supplemented with 0.15% w/v BSA (MilliporeSigma) to remove any residual cytokines. hPBMC (1×10⁶) were co-cultured with cytokine-stimulated HDBEC monolayers at 37° C. for 20 min. To remove any cells adherent by electrostatic interactions, the monolayers were washed twice with 37° C. medium 199 supplemented with 0.15% w/v BSA. HDBEC monolayers and adherent hPBMC were then fixed in 2% glutaraldehyde (MilliporeSigma) for 15 min and washed twice in PBS. The extent of hPBMC adhesion and transmigration was imaged using phase-contrast microscopy with an inverted bright-field microscope (IX71; Olympus, Tokyo, Japan) at ×20 magnification. A total of 5 images of 5 different views were taken per well and processed offline using Image Pro 7 software (Media Cybernetics, Rockville, MD, USA). hPBMC were manually tagged as being surface adherent (phase bright and rounded) or as having transmigrated (phase dark with altered morphology). Total hPBMC adhesion and mean percentage transmigration were calculated for each well.

Endotoxin-free assay: The administered solutions were confirmed as endotoxin-free by a commercial test kit of Limulus polyphemus lysate assay (<0.01 EU/10 mg).

Statistical analysis: Statistical analysis was performed with Graph-Pad Prism (Graph-Pad Software 8.0). All data are presented as means±S.D. and were analysed using one- or two-way ANOVA followed by Bonferroni's for multiple comparison tests (more than two groups). Differences between means were considered statistically significant when P≤0.05 was achieved. Sample size was chosen to ensure alpha 0.05 and power 0.8.

### Example 8: Neutralizing Monoclonal Antibody Sequences

The monoclonal antibody development carried as described in Example 5 allowed to obtain the neutralizing antibody of the invention (here referred as Ab17-IPL-1).

Full length—heavy chain (HC) and light chain (LC)—sequencing of murine hybridoma cell line antibody was carried out. The anti-interleukin-17A antibody, has a heavy chain (HC) having the amino acid sequence of SEQ ID NO: 3 and a light chain (LC) having the amino acid sequence of SEQ ID NO: 5. The 6 CDR regions, said CDR are:
a. a HC-CDR1 of amino acid sequence of SEQ ID NO: 6;
b. a HC-CDR2 of amino acid sequence of; SEQ ID NO:7;
c. a HC-CDR3 of amino acid sequence of SEQ ID NO:8;
d. a LC-CDR1 of amino acid sequence of SEQ ID NO:9;
e. a LC-CDR2 of amino acid sequence of STS (Ser-Thr-Ser); and
f. a LC-CDR3 of amino acid sequence of SEQ ID NO:10.

### Methods:

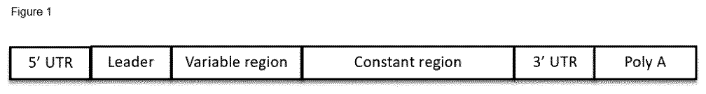

Total RNA was extracted from hybridoma cells and cDNA was subsequently synthesized. Antibody genes were then amplified by isotype-specific PCR, sub-cloned into a standard cloning vector separately and sequenced. The primary structure of antibody-encoding cDNA is illustrated in FIG. 1.

Total RNA was extracted from cultured hybridoma cells (TaKaRa MiniBEST Universal RNA Extraction Kit), cDNA were then synthesized by reverse transcription using oligo-dT primers (PrimeScript RT reagent Kit with gDNA Eraser and TA-cloning Kit (Takara)), and HC (heavy chain) and LC (light chain) were finally amplified by PCR (PacBio RS II sequencer).

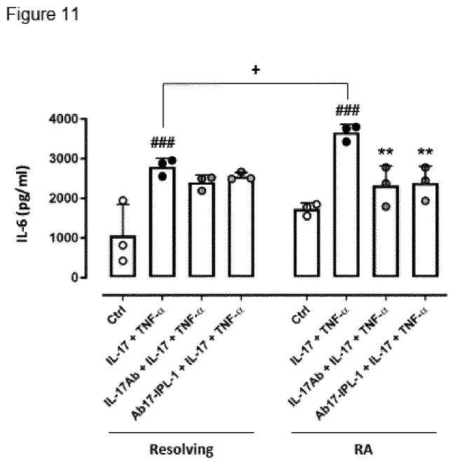

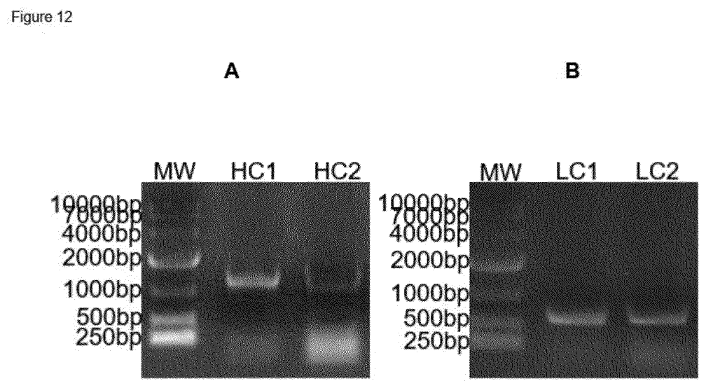

HC and LC, respectively amplified by IgG degenerate primers and Kappa-specific primers, could be observed by gel electrophoresis (FIG. 12), confirming that isotype is IgGKappa. The PCR products were then sub-cloned into a standard vector, followed by bacteria transformation, then colony picking and validation by PCR (FIG. 13), and finally sequencing of 7/8 positive clones for each chain.

### Example 9: Use of the Neutralizing Monoclonal Antibody of the Invention (Ab17-IPL-1) for Treating Inflammatory-Based and Autoimmune Diseases (e.g. Rheumatoid Arthritis and Psoriasis)

Isolation of human fibroblasts: Synovial tissue samples were obtained by ultrasound-guided biopsy from treatment-naive patients with a new onset of clinically apparent arthritis and a symptom duration of ≤12 weeks, who at follow-up had either a resolving arthritis (Res). Patients were classified as having resolving arthritis if there was no clinical evidence of synovial swelling at any peripheral joint (out of a swollen joint count of 66 joints) on final examination at least 1 year after initial presentation, in the absence of disease-modifying antirheumatic drugs (DMARD) or glucocorticoid therapy for at least the previous 3 months. In addition, synovial tissue samples were collected from subjects with established rheumatoid arthritis (RA). RA was classified according to 2010 American College of Rheumatology criteria. Prior to biopsy, the extent of greyscale synovitis and power Doppler enhancement within the synovium of the biopsied joint was systematically graded using a 0-3 scale. Fibroblasts were isolated as previously described (Filer et al., 2011) and used between passages 4 and 65. Fibroblast monolayers were stimulated with IL-17 (10 ng/ml) and TNF-α (100 U/ml) alone or in combination (30 minutes before) with a commercial IL-17Ab neutralizing monoclonal antibody (MAB421) or the antibody of the invention Ab17-IPL-1 at concentration of 10 μg/ml for 24 h at 37° C. Supernatants were collected and measured to evaluate IL-6 levels.

All human samples were obtained with written, informed consent and approval from the Human Biomaterial Resource Centre (Birmingham, UK), West Midlands and Black Country Research Ethics Committee, North East Tyne and West South Research Ethics Committee, or University of Birmingham Local Ethical Review Committee in compliance with the Declaration of Helsinki.

Elisa assay: The levels of IL-6 were measured using commercially available enzyme-linked immunosorbent assay kits (ELISA kit, R&D System, Milan, Italy) according to the manufacturer instructions. Briefly, 100 μl of supernatants, diluted standards, quality controls, and dilution buffer (blank) were applied on a plate with the monoclonal antibody for 2 h. After washing, 100 μl of biotin-labeled antibody was added for 1 h. The plate was washed and 100 μl of the streptavidin-HRP conjugate was incubated for a further 30 min period in the dark. The addition of 100 μl of the substrate and stop solution represented the last steps before the reading of absorbance (measured at 450 nm) on a microplate reader. Antigen levels in the samples were determined using a standard curve expressed as pg/ml.

Endotoxin-free assay: The administered solutions were confirmed as endotoxin-free by a commercial test kit of Limulus polyphemus lysate assay (<0.01 EU/10 mg).

Statistical analysis: Statistical analysis was performed with Graph-Pad Prism (Graph-Pad Software 8.0). Multivariant data were analysed using analysis of variance with Bonferroni post-test or Kruskal-Wallis test with Dunn post-test. p<0.05 was considered as statistically significant.

Results: Data obtained allowed to conclude that the antibody of the invention (Ab17-IPL-1) ameliorates human rheumatoid arthritis (RA) fibroblast activation: Stimulation of fibroblasts from both Res and RA conditions with a combination of IL-17 (10 ng/ml) and TNF-α (100 U/ml) induced the release of IL-6 (means values: 2795.27 μg/ml for Res and 3662.05 μg/ml for RA).

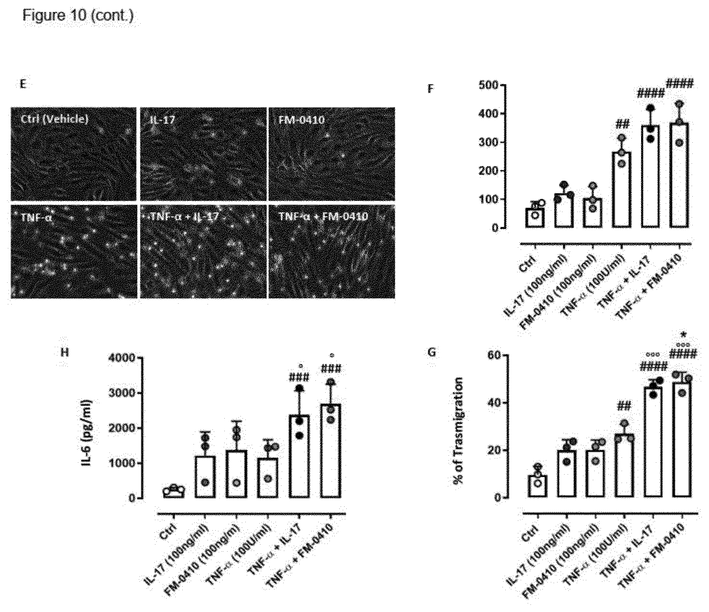

Notably, in the presence of Ab17-IPL-1 we observed a significant reduction of IL-6 production (2383.61 μg/ml) in a similar trend compared to MAB421 antibody (2316.19 μg/ml). No significant effects were observed for both neutralizing antibodies in Res conditions (FIG. 11).

### Example 10: In Vivo Neutralisation and Immunogenicity Assays

For the neutralisation assay, CD-1 mice (n=5 per group) were injected i.p. with 100 μg of IL-17 neutralising antibodies (bimekizumab, secukinumab, MAB317 or Ab-IPL-IL-17™) 30 min prior to an i.p. injection of 10 μg IL-17A or IL-17F (2057-IL, R&D System). 2 h after IL-17s administration, IL-17A and IL-17F levels were determined by Elisa. For the evaluation of immunogenic effects CD-1 mice (n=5 per group) were injected i.p. with 100 μg of IgG1 isotype antibody (vehicle) or IL-17 neutralising antibodies (bimekizumab, secukinumab, MAB317 or Ab-IPL-IL-17™). In a selected time-point of 2 h, 24 h, 72 h, 7 days, 14 days and 21 days total IgG and IgG1 levels were determined by Elisa. The route, timing, and frequency of administration as well as the selected dosages of tested compounds were selected according to updated literature.

### Results:

Data obtained allowed to conclude that the antibody of the invention (Ab17-IPL-1) is able to neutralize IL-17A, 11-17 and IL-17A/F heterodimer in a more prominent wat compared to reference and gold standard antibodies.

### Example 11. AIA Model

Animal studies were regulated by the Animals (Scientific Procedures) Act 1986 of the United Kingdom and performed under appropriate Personal Project License. Approval was granted by the University of Birmingham's Animal Welfare and Ethical Review Body and all ethical guidelines were adhered to whilst carrying out this study. Eight-week-old male, C57Bl/6J wild type (WT) mice were purchased from Charles River and were maintained in a specific pathogen free facility, with free access to food and water. Environmental conditions were: 21±2° C., 55±10% relative humidity and a 12 h light-dark cycle. Mice were immunised with methylated bovine serum albumin (mBSA, 10 μg subcutaneous [s.c.], Sigma-Aldrich) in complete Freund's adjuvant (CFA, Thermofisher scientific, Milan, Italy). On day 21, monoarthritis was induced by intraarticular injection of mBSA (100 μg) into the knee. Mice were treated therapeutically at 24 h or 72 h post disease onset by intraperitoneal (i.p.) injection with 50 μg of either infliximab (anti-TNF-α) or a neutralising antibody to IL-17 (Ab-IPL-IL-17™). Joint thickness (mm) was measured by callipers daily for up to 7 days. Data are expressed as a percentage change from baseline measurement taken on day 21 or area under the curve (AUC).

### Results:

Data obtained allowed to conclude that the antibody of the invention (Ab17-IPL-1) is able to ameliorate onset and progression of mouse pathology in a more extensive manner compared to gold standard antibody.

### Example 12: Haematological Investigations

Standard laboratory procedures were used for blood sampling and measurements (95). Haematological investigations, for all experimental conditions, including blood count test, leukocyte, and sidereal formula were performed on citrated and not-anticoagulated blood samples, respectively. Serological tests were performed by CELL-DYN Sapphire purchased from Abbott S.R.L. (Milan, Italy). All procedures were conducted under strictly aseptic conditions.

TABLE 1
Haematological parameters of vehicle, Ab-IPL-IL-17 ™, MAB421 and secukinumab- treated
mice. Serum samples collected by intracardiac puncture of vehicle, Ab-IPL-IL-17 ™, MAB421
or secukinumab (100 μg/mouse)- treated mice were assessed for haematological parameters
(WBC, MID, GRA, RBC, HGB, HCT, MCV, MCH, MHCH, RDW, MPV, PCT, PWD) at indicated time-points.
Results obtained were expressed as the mean ± SD. Statistical analysis was performed
by using one-way ANOVA followed by Bonferroni’s for multiple comparisons.

| | Vehicle | Ab-IPL-IL-17 ™ | MAB421 | Secukinumab | Bimekizumab |
|---|---|---|---|---|---|
| | (n = 5) | (n = 5) | (n = 5) | (n = 5) | (n = 5) |
| WBC^{††} 10³/μL | | | | | |
| 2 h | 3.60 ± 1.43 | 3.78 ± 1.29 | 3.84 ± 1.30 | 3.96 ± 1.19 | 3.90 ± 0.91 |
| 24 h | 3.52 ± 0.91 | 4.06 ± 1.70 | 4.08 ± 1.22 | 4.44 ± 1.88 | 4.60 ± 1.70 |
| 72 h | 3.38 ± 1.15 | 4.48 ± 1.99 | 4.56 ± 2.25 | 5.10 ± 2.34 | 4.84 ± 2.49 |
| 7 d | 3.60 ± 1.29 | 4.02 ± 1.90 | 4.08 ± 1.77 | 4.74 ± 1.30 | 5.08 ± 1.37 |
| 14 d | 3.60 ± 0.83 | 3.88 ± 0.81 | 3.70 ± 1.30 | 3.08 ± 0.86 | 3.00 ± 0.68 |
| 21 d | 3.34 ± 0.94 | 3.44 ± 1.40 | 3.30 ± 1.58 | 3.280 ± 1.47 | 3.32 ± 1.18 |
| MID^{††} 10³/μL | | | | | |
| 2 h | 0.34 ± 0.23 | 0.38 ± 0.19 | 0.36 ± 0.23 | 0.38 ± 0.31 | 0.36 ± 0.24 |
| 24 h | 0.34 ± 0.21 | 0.48 ± 0.19 | 0.46 ± 0.13 | 0.44 ± 0.25 | 0.42 ± 0.31 |
| 72 h | 0.38 ± 0.15 | 0.56 ± 0.23 | 0.54 ± 0.21 | 0.40 ± 0.25 | 0.42 ± 0.22 |
| 7 d | 0.34 ± 0.25 | 0.46 ± 0.09 | 0.42 ± 0.26 | 0.40 ± 0.16 | 0.46 ± 0.18 |
| 14 d | 0.38 ± 0.19 | 0.44 ± 0.11 | 0.40 ± 0.16 | 0.38 ± 0.13 | 0.40 ± 0.10 |
| 21 d | 0.34 ± 0.22 | 0.36 ± 0.11 | 0.36 ± 0.17 | 0.34 ± 0.15 | 0.32 ± 0.16 |
| GRA^{††} 10³/μL | | | | | |
| 2 h | 0.20 ± 0.16 | 0.22 ± 0.19 | 0.20 ± 0.16 | 0.22 ± 0.16 | 0.24 ± 0.11 |
| 24 h | 0.20 ± 0.10 | 0.26 ± 0.15 | 0.26 ± 0.13 | 0.24 ± 0.11 | 0.22 ± 0.16 |
| 72 h | 0.22 ± 0.11 | 0.38 ± 0.08 | 0.34 ± 0.11 | 0.30 ± 0.16 | 0.30 ± 0.10 |
| 7 d | 0.20 ± 0.10 | 0.28 ± 0.08 | 0.28 ± 0.13 | 0.24 ± 0.13 | 0.26 ± 0.11 |
| 14 d | 0.18 ± 0.08 | 0.26 ± 0.11 | 0.24 ± 0.13 | 0.20 ± 0.10 | 0.20 ± 0.12 |
| 21 d | 0.20 ± 0.10 | 0.24 ± 0.05 | 0.24 ± 0.11 | 0.20 ± 0.10 | 0.22 ± 0.13 |
| RBC^{††} 10⁶/μL | | | | | |
| 2 h | 5.99 ± 1.29 | 5.74 ± 1.22 | 5.56 ± 1.12 | 5.57 ± 1.18 | 5.84 ± 1.62 |
| 24 h | 6.09 ± 1.203 | 5.96 ± 1.05 | 5.66 ± 1.21 | 4.48 ± 1.11 | 4.63 ± 0.99 |
| 72 h | 6.12 ± 1.02 | 6.30 ± 0.78 | 6.29 ± 1.19 | 3.7 ± 1.05* | 3.79 ± 1.02* |
| 7 d | 6.14 ± 1.19 | 6.29 ± 0.95 | 6.09 ± 0.66 | 4.10 ± 0.78 | 4.18 ± 0.93 |
| 14 d | 6.12 ± 1.03 | 6.47 ± 1.00 | 6.12 ± 0.46 | 5.98 ± 1.42 | 5.81 ± 1.41 |
| 21 d | 6.15 ± 0.98 | 6.41 ± 0.96 | 6.27 ± 0.54 | 6.25 ± 1.18 | 6.37 ± 1.11 |
| HGB^{††} g/dL | | | | | |
| 2 h | 9.84 ± 2.22 | 10.58 ± 1.55 | 10.10 ± 1.87 | 9.92 ± 1.78 | 9.88 ± 1.02 |
| 24 h | 10.08 ± 1.74 | 10.72 ± 1.50 | 10.26 ± 1.62 | 8.88 ± 1.65 | 9.08 ± 1.55 |
| 72 h | 10.20 ± 1.73 | 10.84 ± 1.56 | 10.26 ± 1.71 | 6.92 ± 1.53* | 7.10 ± 1.38* |
| 7 d | 10.32 ± 1.366 | 10.74 ± 1.03 | 10.20 ± 1.17 | 7.76 ± 1.31 | 8.10 ± 1.33 |
| 14 d | 10.28 ± 1.593 | 10.96 ± 1.23 | 10.34 ± 1.30 | 9.480 ± 1.36 | 9.06 ± 1.84 |
| 21 d | 10.22 ± 1.897 | 10.90 ± 1.06 | 10.42 ± 1.08 | 10.16 ± 1.52 | 10.06 ± 1.27 |
| HCT^{††} % | | | | | |
| 2 h | 30.24 ± 6.13 | 29.84 ± 4.21 | 28.62 ± 3.68 | 29.56 ± 2.26 | 29.96 ± 2.29 |
| 24 h | 30.30 ± 5.25 | 30.24 ± 3.57 | 28.98 ± 3.97 | 28.86 ± 1.80 | 29.56 ± 2.16 |
| 72 h | 31.00 ± 5.00 | 31.64 ± 3.64 | 30.28 ± 4.38 | 21.30 ± 5.47** | 22.04 ± 4.37** |
| 7 d | 30.58 ± 4.76 | 31.46 ± 2.97 | 29.58 ± 4.05 | 22.94 ± 4.68* | 22.48 ± 3.74* |
| 14 d | 30.70 ± 5.35 | 31.86 ± 1.87 | 30.48 ± 1.80 | 27.88 ± 2.63 | 28.36 ± 3.19 |
| 21 d | 31.00 ± 4.61 | 31.88 ± 2.12 | 31.16 ± 2.23 | 29.04 ± 2.30 | 29.20 ± 2.70 |
| MCV^{††} fL | | | | | |
| 2 h | 62.96 ± 10.27 | 60.62 ± 8.00 | 60.76 ± 7.5 | 61.86 ± 8.95 | 61.22 ± 5.70 |
| 24 h | 64.16 ± 10.23 | 62.32 ± 8.25 | 61.22 ± 6.33 | 63.60 ± 8.78 | 62.32 ± 10.33 |
| 72 h | 64.24 ± 11.81 | 62.98 ± 8.30 | 61.34 ± 8.01 | 81.12 ± 8.84 | 80.98 ± 9.17 |
| 7 d | 63.06 ± 9.67 | 62.60 ± 9.27 | 62.00 ± 6.18 | 71.98 ± 8.64 | 72.78 ± 8.77 |
| 14 d | 63.12 ± 7.37 | 62.62 ± 8.51 | 60.76 ± 5.23 | 63.46 ± 8.13 | 61.10 ± 9.83 |
| 21 d | 64.10 ± 8.72 | 63.18 ± 8.74 | 62.36 ± 4.85 | 62.56 ± 7.90 | 62.92 ± 6.53 |
| MCH^{††} pg | | | | | |
| 2 h | 16.88 ± 1.83 | 17.48 ± 1.32 | 17.52 ± 1.06 | 17.58 ± 1.42 | 16.98 ± 1.38 |
| 24 h | 17.28 ± 2.25 | 17.32 ± 1.75 | 17.90 ± 1.38 | 19.10 ± 1.88 | 18.92 ± 1.42 |
| 72 h | 17.40 ± 2.02 | 17.70 ± 1.44 | 17.56 ± 1.12 | 21.78 ± 4.20* | 21.46 ± 2.04* |
| 7 d | 17.42 ± 1.72 | 17.54 ± 1.19 | 17.60 ± 0.62 | 21.14 ± 4.82 | 21.22 ± 3.74 |
| 14 d | 17.76 ± 1.77 | 17.74 ± 1.18 | 17.44 ± 0.70 | 17.50 ± 1.09 | 17.48 ± 1.38 |
| 21 d | 17.04 ± 1.66 | 17.08 ± 1.18 | 17.40 ± 0.95 | 17.62 ± 1.05 | 17.96 ± 1.57 |
| MHCH^{††} g/dL | | | | | |
| 2 h | 30.48 ± 3.57 | 29.94 ± 3.12 | 30.76 ± 4.19 | 30.60 ± 4.27 | 30.00 ± 3.06 |
| 24 h | 30.80 ± 3.80 | 29.52 ± 3.76 | 30.74 ± 3.74 | 31.54 ± 4.15 | 30.70 ± 3.18 |
| 72 h | 31.14 ± 3.94 | 30.66 ± 3.54 | 30.28 ± 4.05 | 30.90 ± 6.72 | 30.62 ± 5.57 |
| 7 d | 30.64 ± 3.74 | 29.99 ± 2.86 | 30.50 ± 5.29 | 29.50 ± 5.03 | 29.36 ± 5.05 |
| 14 d | 30.82 ± 3.00 | 30.72 ± 2.61 | 30.34 ± 4.24 | 29.64 ± 4.38 | 29.96 ± 4.45 |
| 21 d | 30.46 ± 4.04 | 30.18 ± 3.55 | 30.00 ± 4.85 | 30.34 ± 4.54 | 30.50 ± 4.52 |
| RDW^{††} % | | | | | |
| 2 h | 17.94 ± 2.17 | 18.08 ± 2.84 | 18.06 ± 2.47 | 18.14 ± 1.87 | 18.82 ± 1.75 |
| 24 h | 17.72 ± 2.03 | 18.04 ± 2.91 | 18.54 ± 1.56 | 18.24 ± 1.12 | 18.42 ± 1.18 |
| 72 h | 18.08 ± 2.61 | 16.90 ± 2.08 | 16.96 ± 1.55 | 16.86 ± 3.47 | 17.12 ± 2.05 |
| 7 d | 18.30 ± 2.72 | 17.94 ± 1.94 | 17.88 ± 1.33 | 18.38 ± 1.90 | 18.14 ± 3.00 |
| 14 d | 18.06 ± 3.03 | 17.86 ± 1.76 | 17.76 ± 1.90 | 18.06 ± 2.06 | 18.26 ± 2.28 |
| 21 d | 18.40 ± 2.50 | 17.88 ± 1.94 | 17.60 ± 0.58 | 18.34 ± 1.33 | 18.02 ± 1.34 |
| MPV^{††} fL | | | | | |
| 2 h | 4.88 ± 0.64 | 4.74 ± 0.56 | 4.66 ± 0.50 | 4.54 ± 0.5771 | 4.64 ± 0.86 |
| 24 h | 4.92 ± 0.57 | 4.82 ± 0.53 | 4.96 ± 0.68 | 5.16 ± 0.8764 | 5.00 ± 1.12 |
| 72 h | 4.98 ± 0.52 | 4.96 ± 0.53 | 5.08 ± 0.58 | 7.80 ± 1.733**** | 7.96 ± 1.95**** |
| 7 d | 4.88 ± 0.62 | 5.00 ± 0.67 | 4.84 ± 0.54 | 6.58 ± 1.242* | 6.78 ± 1.09* |
| 14 d | 4.90 ± 0.50 | 5.12 ± 0.67 | 4.98 ± 0.57 | 5.86 ± 0.5128 | 5.66 ± 0.92 |
| 21 d | 5.04 ± 0.55 | 5.12 ± 0.40 | 4.92 ± 0.73 | 4.92 ± 0.4207 | 5.02 ± 0.76 |
| PCT^{††} % | | | | | |
| 2 h | 0.27 ± 0.09 | 0.28 ± 0.08 | 0.28 ± 0.08 | 0.27 ± 0.08 | 0.26 ± 0.07 |
| 24 h | 0.28 ± 0.08 | 0.28 ± 0.07 | 0.28 ± 0.07 | 0.25 ± 0.09 | 0.26 ± 0.06 |
| 72 h | 0.28 ± 0.10 | 0.29 ± 0.05 | 0.28 ± 0.07 | 0.16 ± 0.06 | 0.17 ± 0.03 |
| 7 d | 0.28 ± 0.09 | 0.28 ± 0.05 | 0.28 ± 0.07 | 0.21 ± 0.05 | 0.22 ± 0.07 |
| 14 d | 0.28 ± 0.10 | 0.28 ± 0.05 | 0.29 ± 0.06 | 0.25 ± 0.05 | 0.26 ± 0.05 |
| 21 d | 0.29 ± 0.07 | 0.28 ± 0.05 | 0.28 ± 0.07 | 0.28 ± 0.04 | 0.28 ± 0.03 |
| PWD^{††} % | | | | | |
| 2 h | 15.98 ± 2.63 | 16.24 ± 2.34 | 16.28 ± 2.31 | 16.30 ± 2.15 | 16.80 ± 1.42 |
| 24 h | 15.86 ± 2.77 | 16.58 ± 2.61 | 16.36 ± 2.37 | 16.06 ± 2.30 | 16.04 ± 1.30 |
| 72 h | 16.02 ± 2.52 | 16.30 ± 1.98 | 16.36 ± 2.25 | 18.02 ± 2.61 | 17.94 ± 2.99 |
| 7 d | 15.76 ± 2.52 | 16.52 ± 2.24 | 16.34 ± 2.40 | 15.54 ± 2.91 | 16.04 ± 2.71 |
| 14 d | 16.04 ± 2.68 | 16.72 ± 1.73 | 16.28 ± 1.98 | 15.98 ± 2.03 | 16.02 ± 1.63 |
| 21 d | 16.10 ± 2.37 | 16.78 ± 1.49 | 16.24 ± 2.35 | 16.14 ± 1.80 | 16.24 ± 1.83 |

*P ≤ 0.05, **P ≤ 0.01, ****P ≤ 0.0001 vs Vehicle group; (n = 5 mice per group).
^{††}mean ± SD; WBC = White Blood Cells; MID = Minimum Inhibitory Dilution; GRA = Granulocytes; RBC = Red Blood Cells; HGB = Hemoglobin; HCT = Hematocrit; MCV = Mean Cell Volume; MCH = Mean Corpuscular Hemoglobin; MCHC = Mean Corpuscular Hemoglobin Concentration; RDW = Red blood cells Distribution Width; MPV = Mean Platelet Volume; PCT = Plateletcrit; PWD = Platelet Distribution Width.

### Results:

Data obtained allowed to conclude that the antibody of the invention (Ab17-IPL-1) display less off-target effects and minor immunogenicity compared to reference and gold standard antibodies.

### Materials and Methods

### Ex Vivo Analysis

### In Vivo Neutralisation and Immunogenicity Assays

For the neutralisation assay, CD-1 mice (n=5 per group) were injected i.p. with 100 μg of IL-17 neutralising antibodies (secukinumab, MAB317 or Ab-IPL-IL-17™), 30 mi prior to an i.p. injection of 10 μg IL-17A or IL-17F (2057-IL, R&D System). 2 h after IL-17s administration, IL-17A and IL-17F levels were determined by Elisa. For the evaluation of immunogenic effects CD-1 mice (n=5 per group) were injected i.p. with 100 μg of IgG1 isotype antibody (vehicle) or IL-17 neutralising antibodies (secukinumab, MAB317 or Ab-IPL-IL-17™). In a selected time-point of 2 h, 24 h, 72 h, 7 days, 14 days and 21 days total IgG and IgG1 levels were determined by Elisa. The route, timing, and frequency of administration as well as the selected dosages of tested compounds were selected according to updated literature (Raucci F, Iqbal A J, Saviano A, et al. IL-17A neutralizing antibody regulates monosodium urate crystal-induced gouty inflammation. Pharmacol Res 2019; 147:104351 and Raucci F, Saviano A, Casillo G M, et al. IL-17-induced inflammation modulates the mPGES-1/PPAR-γ pathway in monocytes/macrophages. Br J Pharmacol 2022; 179(9):1857-73).

### Ex Vivo Whole Blood Assay

Whole blood culture and stimulation were performed as previously described with small modifications (Papandreou V, Kavrochorianou N, Katsoulas T, et al. Adrenergic Effect on Cytokine Release After Ex Vivo Healthy Volunteers' Whole Blood LPS Stimulation. Inflammation 2016; 39(3):1069-75). Briefly, venous blood collected into lithium heparin tubes from patients with clinically diagnosed IBD was placed into 96-well plates with or without Ab-IPL-IL-17™ (10 μg/ml) at 37° C., 5% CO₂ for 4 h. Following incubation, samples were centrifuged (300 g, 5 min at RT), and supernatants were collected and stored at −80° C. until IL-17A levels measurements by Elisa assay.

### Elisa and Elisa Spot Assays

The levels of IL-6 (DY406 and DY206, respectively, mouse and human kits) and TNF-α (DY210) in the in vitro and ex vivo supernatants were measured at 2 h or 24 h using commercially available enzyme-linked immunosorbent assay kits (Elisa kit, R&D System) according to the procedure previously described (Raucci F, Saviano A, Casillo G M, et al. IL-17-induced inflammation modulates the mPGES-1/PPAR-γ pathway in monocytes/macrophages. Br J Pharmacol 2022; 179(9):1857-73). Briefly, 100 μl of supernatants, diluted standards, quality controls, and dilution buffer (blank) were applied on a plate with the monoclonal antibody for 2 h. After washing, 100 μl of biotin-labelled antibody was added, and incubation continued for 1 h. The plate was washed, and 100 μl of the streptavidin-HRP conjugate was added, and the plate was incubated for a further 30 min period in the dark. The addition of 100 μl of the substrate and stop solution represented the last steps before the reading of absorbance (measured at 450 nm) on a microplate reader. Antigen levels in the samples were determined using a standard curve and expressed as pg/ml. Similarly, serum level of IL-17A, IL-17F, total IgG and IgG1 (Elisa kit, R&D System) were measured at indicated time-points for in vivo neutralisation and immunogenicity assays.

Inflammatory exudates from the air pouch were incubated with the precoated proteome profiler array membranes according to the manufacturer's instructions (ARY006, R&D System). Dot plots were detected using the enhanced chemiluminescence detection kit and Image Quant 400 GE Healthcare software (GE Healthcare, Italy) and successively quantified using GS 800 imaging densitometer software (Bio-Rad) as extensively described (Cristiano C, Volpicelli F, Lippiello P, et al. Neutralization of IL-17 rescues amyloid-β-induced neuroinflammation and memory impairment. Br J Pharmacol 2019; 176(18):3544-57 and Saviano A, Casillo G M, Raucci F, et al. Supplementation with ribonucleotide-based ingredient (Ribodiet®) lessens oxidative stress, brain inflammation, and amyloid pathology in a murine model of Alzheimer. Biomed Pharmacother 2021; 139:111579).

### Haematological Investigations

Standard laboratory procedures were used for blood sampling and measurements. Haematological investigations, for all experimental conditions, including blood count test, leukocyte, and sidereal formula were performed on citrated and not-anticoagulated blood samples, respectively. Serological tests were performed by CELL-DYN Sapphire purchased from Abbott S.R.L. (Milan, Italy). All procedures were conducted under strictly aseptic conditions.

### Circular Dichroism

CD experiments were performed on a Jasco J-815 spectropolarimeter equipped with a PTC-423S/15 Peltier temperature controller. Cells with 0.1 cm path length and peptide concentration of 0.1 mM were used to record CD spectra between 200 and 260 nm, with a 1 nm bandwidth and a scan rate of 20 nm/min. Spectra were recorded at 20° C. Spectra were signal-averaged over three scans, baseline-corrected by subtracting a buffer spectrum, and smoothed using the means-movement function. Spectra were analyzed for secondary structure composition using the BeStSel method.

### Computational Studies

nIL-17™ 3D Structure Prediction

PEP-FOLD 4 webserver (Lamiable A, Thévenet P, Rey J, et al. PEP-FOLD3: faster de novo structure prediction for linear peptides in solution and in complex. Nucleic Acids Res 2016; 44(W1):W449-54, Tufféry P, Derreumaux P. A refined pH-dependent coarse-grained model for peptide structure prediction in aqueous solution. Front Bioinform 2023; 3:1113928 and Binette V, Mousseau N, Tuffery P. A Generalized Attraction-Repulsion Potential and Revisited Fragment Library Improves PEP-FOLD Peptide Structure Prediction. J Chem Theory Comput 2022; 18(4):2720-36) was used to predict the nIL-17™ structure from the amino acid sequence of the peptide. The primary sequence is submitted using the single letter code and the program predicts the 3D conformation by assembling predicted conformations of short local sequences using a greedy procedure driven by a coarse-grained energy score. PEP-FOLD 4 uses a new version of the force field (sOPEP2) that makes use of a Mie representation instead of the former Van der Waals representation for non-bonded interactions, and also includes an energy term using the Debye-Hueckel formalism to model pH, ionic strength dependence, and extremity blocking.

### Molecular Docking

All docking calculations were conducted using the Schrödinger molecular modeling suite (version 2021-4). Protein structures were obtained from the Protein Data Bank (PDB). In particular, the structure of the complex of IL-17A (monomers A and B) with IL-17RA and IL-17RC receptors (PDB id: 7ZAN) (Goepfert A, Barske C, Lehmann S, et al. IL-17-induced dimerization of IL-17RA drives the formation of the IL-17 signalosome to potentiate signaling. Cell Rep 2022; 41(3):111489) was selected to perform molecular docking studies with nIL-17™ peptide. The IL-17RA and IL-17RC receptors and nIL-17™ were prepared with the aid of the Protein Preparation Wizard panel of Maestro Suite (Sastry G M, Adzhigirey M, Day T, et al. Protein and ligand preparation: parameters, protocols, and influence on virtual screening enrichments. J Comput Aided Mol Des 2013; 27(3):221-34) adding the missing hydrogen atoms and removing any water molecule with less than two hydrogen bonds to non-water molecules. In addition, the side chain ionization and tautomeric states were predicted, and the H-bonding network of the receptor refined minimizing the position of each hydrogen. Receptor grid generation tool of Glide software was used to generate the search grid around the IL-17A C-terminal region of monomers A and B to perform docking simulations of IL-17RA and IL-17RC, respectively. Then, docking calculations were performed using Glide 9.4 in its SP-peptide variant and employing the OPLS4 force field. Thus, the top-ranked complexes were selected and visually checked for a good chemical geometry.

From the above description and the above-noted examples, the advantage attained by the product described and obtained according to the present invention are apparent.

1. An isolated anti-interleukin-17A antibody, wherein said antibody comprises:
a. a heavy chain (HC) having the amino acid sequence of SEQ ID NO: 3;
and
b. a light chain (LC) having the amino acid sequence of SEQ ID NO: 5.

2. The isolated anti-interleukin-17A antibody of claim 1, wherein said antibody comprises 6 CDR regions, said CDR regions being:
a. a HC-CDR1 having the amino acid sequence of SEQ ID NO: 6;
b. a HC-CDR2 having the amino acid sequence of; SEQ ID NO:7;
c. a HC-CDR3 having the amino acid sequence of SEQ ID NO:8;
d. a LC-CDR1 having the amino acid sequence of SEQ ID NO:9;
e. a LC-CDR2 having the amino acid sequence of STS (Ser-Thr-Ser); and
f. a LC-CDR3 having the amino acid sequence of SEQ ID NO:10.

3. The isolated anti-interleukin-17A antibody according to claim 1, wherein said antibody is a monoclonal antibody, a chimeric antibody and/or is humanized or human.

4. The antibody according to claim 1, wherein said antibody binds to the peptide of SEQ ID NO:1.

5. A polynucleotide encoding the antibody according to claim 1.

6. A vector comprising the polynucleotide of claim 5, wherein the vector is optionally an expression vector.

7. A host cell comprising the vector of claim 6, wherein said host cell is prokaryotic, eukaryotic, or mammalian.

8. A pharmaceutical composition comprising (i) the anti-interleukin-17A antibody according to claim 1, wherein the composition optionally further comprises pharmaceutically acceptable carriers and/or excipients.

9. (canceled)

10. A method of treating psoriasis, psoriatic arthritis (PsA), rheumatoid arthritis (RA), or ankylosing spondylitis (AS) in a patient in need thereof, said method comprising
administering to said patient a pharmaceutical effective amount of the antibody according to claim 1.

11. A method for the identification of anti-interleukin-17 compounds, said method comprising the step of using the peptide according to claim 4.
